(19) United States
(12) Patent Application Publication
(10) Pub. No.: US 2006/0218667 A1
(43) Pub. Date: Sep. 28, 2006

(54) Plant-produced recombinant aprotinin and aprotinin variants

(76) Inventors: Fakhrieh S. Vojdani, Davis, CA (US); Kenneth E. Palmer, Vacaville, CA (US); Stephen J. Garger, Vacaville, CA (US); Gregory P. Pogue, Vacaville, CA (US)

Correspondence Address: LARGE SCALE BIOLOGY CORPORATION, 3333 VACA VALLEY PARKWAY, SUITE 1000, VACAVILLE, CA, 95688, US

(21) Appl. No.: 11/249,685

(22) Filed: Oct. 12, 2005

Related U.S. Application Data
(60) Provisional application No. 60/618,485, filed on Oct. 12, 2004.
(60) Provisional application No. 60/635,214, filed on Dec. 10, 2004.

(51) Int. Cl. A01H 5/00; C07H 21/04; C12N 15/82; C12N 5/04; A01H 1/00; C07K 14/415

(52) U.S. Cl. 800/280; 435/419; 435/468; 536/23.6; 530/370; 977/804

(57) ABSTRACT

The present invention relates to plant produced native aprotinin and aprotinin variants having enzyme-inhibitory, immunological and pharmacokinetic properties and their preparation. In a preferred method a recombinant RNA plant virus is used to express native aprotinin+variants thereof in Nicotiana plants.

### CROSS REFERENCE TO RELATED APPLICATIONS

This application claims the benefit of U.S. Provisional Application No. 60/618,485, filed on Oct. 12, 2004 and U.S. Provisional Application No. 60/635,214, filed on Dec. 10, 2004, which are both incorporated herein by reference.

### FIELD OF USE

This invention relates to plant-produced recombinant bovine lung aprotinin, variants thereof, and related methods. In addition, the present invention relates to plant viral vectors which are (a) self-replicating; (b) capable of systemic infection in a host; (c) contain, or are capable of containing, nucleic acid sequences foreign to the native virus, which are transcribed or expressed in the host plant; and (d) stable, especially for the transcription and expression of foreign nucleic acid sequences, such as that encoding aprotinin and certain variants thereof.

### BACKGROUND OF THE INVENTION

The publications and other materials referred to herein to describe the background of the invention and to provide additional detail with regard to the practice of this invention are incorporated herein by reference.

Aprotinin is a serine proteinase inhibitor (“serpin”), which modulates the inflammatory responses associated with use of cardiopulmonary bypass procedures. It consists of 58 amino acid residues in a single chain, cross-linked by 3 disulphide bridges, with a total molecular weight of 6512.

The mechanism of action is complex, affecting the extrinsic and intrinsic coagulation pathways at several levels via inhibition of kallikrein and plasmin mediators. When administered as a loading dose followed by continuous infusion and with a pump-prime dose in the bypass circuit, these effects combine to reduce perioperative blood loss and the need for replacement blood products in cardiac bypass surgery.

Aprotinin (bovine origin) (Bayer's Trasylol®) is an FDA approved product indicated for prophylactic use to reduce perioperative blood loss and the need for blood transfusion in patients undergoing cardiopulmonary bypass in the course of coronary artery bypass graft surgery (CABG). Clinical studies currently under way on the benefits of aprotinin in other indications, both prophylactic and therapeutic, where the control of pathophysiological inflammatory cascades is desirable, suggest that the market for aprotinin could expand significantly.

Because the only current source of research grade Aprotinin or of active pharmaceutical ingredient (API) is bovine tissue, a source that has experienced supply constraints in the past even for the current label indication, there is a need to develop and commercialize a finished product containing an active ingredient that is reliably produced, consistent in supply and quality, and not subject to concerns over animal-associated adventitious agents such as bovine spongiform encephalopathy (BSE). In addition, because the FDA-approved aprotinin product is derived from bovine lung, impurities and contaminants of animal origin pose potential risks to patients. Of particular concern currently is the prion causing bovine spongioform encephalopathy, which can be transmitted to humans and cause variant Creutzfeld-Jacob disease.

The recombinant plant industry has experienced contamination of food/feed crops with transgenic crops; therefore, there is a need to produce Aprotinin in a plant that will not contaminate food/feed crops. Therefore, Applicants have developed a process for manufacturing recombinant Aprotinin (r-Aprotinin) that is chemically identical to aprotinin (bovine source) in Nicotiana plants. Applicants' manufacturing system uses non-transgenic plant-based production of r-Aprotinin. No animal-sourced raw materials or animal-derived components of any kind are used in the manufacture of this recombinant molecule. Plants have never been reported to harbor infectious agents for human or animal hosts. The process involves expression of the native, bovine-sequence aprotinin gene in a plant virus vector and production and extraction of the protein using non-food/feed, non-GMO plants. Applicants' biomanufacturing technology yields r-Aprotinin that is identical in amino acid composition, sequence, and specific activity to the bovine lung-derived native aprotinin, suggesting that the protein structures of r-Aprotinin are also identical to the Trasylol® bovine-derived aprotinin.

r-Aprotinin is a monomeric polypeptide composed of 58 amino acids, with a molecular weight of 6,512 Daltons. Applicants noticed that plant produced r-Aprotinin is oxidized to varying degrees at a methionine at amino acid 52. The current FDA-approved aprotinin displays a low level, about 9%, of methionine oxidation. To consistently eliminate such oxidation in r-aprotinin and to eliminate potential safety concerns, Applicants produced in plants r-aprotinin variants that contain a non-methionine amino acid at position 52.

### SUMMARY OF THE INVENTION

The present invention is directed to recombinant plant viral nucleic acids and recombinant viruses which are stable for maintenance and transcription or expression of non-native (foreign) nucleic acid sequences encoding Aprotinin and which are capable of systemically transcribing or expressing such foreign Aprotinin sequences in the host plant. More specifically, recombinant plant viral nucleic acids according to the present invention comprise a native plant viral subgenomic promoter, at least one non-native plant viral subgenomic promoter, a plant viral coat protein coding sequence, and non-native (foreign) nucleic acid sequences encoding Aprotinin.

The invention encompasses a composition comprising a plant produced recombinant variant aprotinin, in which the variant aprotinin has an amino acid other than methionine at position 52. In a preferred embodiment, the amino acid is selected from a group consisting of glutamine, leucine and valine. The composition also is free of microbial and mammalian impurities.

In one embodiment, the invention includes an isolated DNA molecule, comprising
a. a DNA sequence encoding a variant aprotinin, wherein the variant aprotinin has an amino acid other than methionine at position 52; and
b. a DNA sequence encoding an RNA subgenomic promoter;
c. wherein the DNA sequence encoding aprotinin having a oxidation-resistant amino acid at position 52 is attached at its 5′ end to the DNA sequence encoding an RNA subgenomic promoter so that when a resulting negative sense RNA molecule encoded by the DNA molecule is present, then expression of the aprotinin-encoding DNA sequence is allowed in a plant.

In another embodiment an isolated RNA molecule is presented and comprises:
a. a plus sense single stranded RNA subgenomic promoter sequence, and
b. a plus sense single stranded RNA sequence encoding a variant aprotinin, wherein the variant aprotinin has an amino acid other than methionine at position 52;
c. wherein the sequence encoding the variant aprotinin is linked at its 5′ end to said plus sense single stranded RNA subgenomic promoter sequence.

In another embodiment, the invention includes a recombinant single stranded plus sense plant viral RNA, comprising:
a. an RNA coding sequence for 126-kDa and 183-kDa replicase subunits;
b. a first coat protein subgenomic promoter sequence being attached at its 3′ end to an RNA sequence coding for a variant aprotinin, wherein the variant aprotinin has an amino acid other than methionine at position 52.
   In a preferred embodiment, such recombinant single stranded plus sense plant viral RNA, may also include:
a. the RNA coding sequence for 126-kDa and 183-kDa replicase subunits attached at its 3′ end to a 30-kDa open reading frame of a viral movement protein,
c. the 30-kDa open reading frame of a viral movement protein containing the first coat protein subgenomic promoter sequence;
d. the RNA sequence coding for variant aprotinin attached at its 3′ end to a second coat protein subgenomic promoter sequence; and
e. the second coat protein subgenomic promoter sequence attached at its 3′ end to a coat protein coding sequence.

The invention also contemplates a recombinant cDNA plasmid, comprising a phage DNA dependent RNA polymerase promoter operably linked to a cDNA sequence encoding the recombinant single stranded plus sense plant viral RNA as described above.

A host plant cell or a host plant transfected with at least one copy of the above-described recombinant single stranded plus sense plant viral RNA is also presented. Also contemplated is a virus particle made in such host cell and used to infect a plant cell or plant.

The invention also encompasses a plant-compatible expression vector comprising an artificial polynucleotide encoding a recombinant variant aprotinin, wherein the variant aprotinin has an amino acid other than methionine at position 52. In a preferred embodiment, such plant-compatible expression vector is a plant viral vector. A host plant cell containing the above-described expression vector is also contemplated.

Yet another embodiment covers a process for producing a polypeptide comprising a variant aprotinin having an amino acid other than methionine at position 52 by transforming a plant with an appropriate expression vector, as described above, or by infecting a plant with an appropriate viral expression vector.

These and other features and advantages of this invention are described in, or are apparent from, the following detailed description of various exemplary embodiments of the compositions and methods according to this invention.

### BRIEF DESCRIPTION OF THE DRAWINGS

Various exemplary embodiments of this invention will be described in detail, with reference to the following figures.

FIG. 1 is a flow chart showing one method for purifying recombinant aprotinin.

FIG. 2 is a flow chart showing another method for purifying recombinant aprotinin.

FIG. 3 shows an aprotinin analysis and comparison by 16% Tris-Glycine SDS-PAGE and Coomassie Staining.

FIG. 4 shows a comparison of Reversed-Phase HPLC of Trasylol® aprotinin product and of r-Aprotinin.

FIG. 5 shows an overlay of Reversed-Phase HPLC of Trasylol® and of r-Aprotinin.

FIG. 6 is a Coomassie stained SDS-PAGE gel of various purified samples of aprotinin and aprotinin variants. M designates a marker; Trasylol designates Bayer's aprotinin product; Native corresponds to aprotinin product from pLSB2602; Leu corresponds to product from a modified pLSB2602 in which the Met at position 52 has been replaced by Leucine; Val corresponds to product from a modified pLSB2602 in which the Met at position 52 has been replaced by Valine; Gln corresponds to product from a modified pLSB2602 in which the Met at position 52 has been replaced by Glutamine; -Met corresponds to product from a modified pLSB2602 in which the Met at position 52 has been deleted.

FIG. 7 is a graph showing relative expression of native aprotinin and aprotinin variants in Nicotiana excelsiana.

FIG. 8 shows various MALDI-TOF spectra for bovine aprotinin, recombinanat aprotinin, and various aprotinin variants.

### DETAILED DESCRIPTION OF THE INVENTION

r-Aprotinin is a monomeric polypeptide composed of 58 amino acids, with a molecular weight of 6,512 Daltons. The amino acid sequence of r-Aprotinin is as follows:
RPDFCLEPPYTGPCKARIIRYFYNAKAGLCQTFVYGGCRAKRNNFKSAEDCMRT CGGA (SEQ ID NO: 2), where A=alanine, R=arginine, N=asparagines, D=aspartic acid, C=cysteine, E=glutamic acid, Q=glutamine, G=glycine, H=histidine, I=isoleucine, L=leucine, K=lysine, M=methionine, F=phenylalanine, P=proline, S=serine, T=threonine, W=tryptophan, Y=tyrosine, V=valine. The three-dimensional conformation of aprotinin is maintained by three disulfide bridges: Cys₅-Cys₅₅, Cys₁₄-Cys₃₈ and Cys₃₀-Cys₅₁.

As described in detail in the Examples section below, Applicants have produced aprotinin in plants using plant viral expression vectors that comprise artificial polynucleotides that encode aprotinin. In one embodiment the polynucleotide sequence encoding aprotinin is codon optimized for tobacco mosaic virus. In a preferred embodiment the codons are the same as those used in cow, as set forth in SEQ ID NO: 2, amino acid residues #1-58.

Such expression vectors may also encode a signal peptide that directs the newly synthesized protein to the secretory pathway of the cell in which the expression vector is expressed. The sequence encoding the signal peptide is fused in frame with the DNA encoding the polypeptide to be expressed. Signal peptides should be compatible with the expression system corresponding to the expression vector. In a preferred embodiment the signal peptide sequence is extensin from N. benthamiana.

Viral expression vectors encoding r-aprotinin are used to transiently infect plants. A viral expression vector that expresses heterologous proteins in plants preferably includes (1) a native viral subgenomic promoter (Dawson, W. O. et al. (1988) Phytopathology 78:783-789 and French, R. et al. (1986) Science 231:1294-1297), (2) preferably, one or more non-native viral subgenomic promoters (Donson, J. et al. (1991) Proc. Nat. Acad. Sci. USA 88:7204-7208 and Kumagai, M. H. et al. (1993) Proc. Nat. Acad. Sci. USA 90:427-430), (3) a sequence encoding viral coat protein (native or not), and (4) nucleic acid encoding the desired heterologous protein. Vectors that include only non-native subgenomic promoters may also be used. The minimal requirement for the present vector is the combination of a replicase gene and the coding sequence that is to be expressed, driven by a native or non-native subgenomic promoter. The viral replicase is expressed from the viral genome and is required to replicate extrachromosomally. The subgenomic promoters allow the expression of the foreign or heterologous coding sequence and any other useful genes such as those encoding viral proteins that facilitate viral replication, proteins required for movement, capsid proteins, etc. The viral vectors are encapsidated by the encoded viral coat proteins, yielding a recombinant plant virus. This recombinant virus is used to infect appropriate host plants. The recombinant viral nucleic acid can thus replicate, spread systemically in the host plant and direct RNA and protein synthesis to yield the desired heterologous protein in the plant. In addition, the recombinant vector maintains the non-viral heterologous coding sequence and control elements for periods sufficient for desired expression of this coding sequence.

The recombinant viral nucleic acid is prepared from the nucleic acid of any suitable plant virus, though members of the tobamovirus family are preferred. The native viral nucleotide sequences may be modified by known techniques providing that the necessary biological functions of the viral nucleic acid (replication, transcription, etc.) are preserved. As noted, one or more subgenomic promoters may be inserted. These are capable of regulating expression of the adjacent heterologous coding sequences in infected or transfected plant host. Native viral coat protein may be encoded by this RNA, or this coat protein sequence may be deleted and replaced by a sequence encoding a coat protein of a different plant virus (“non-native” or “foreign viral”). A foreign viral coat protein gene may be placed under the control of either a native or a non-native subgenomic promoter. The foreign viral coat protein should be capable of encapsidating the recombinant viral nucleic acid to produce functional, infectious virions. In a preferred embodiment, the coat protein is foreign viral coat protein encoded by a nucleic acid sequence that is placed adjacent to either a native viral promoter or a non-native subgenomic promoter. Preferably, the nucleic acid encoding the heterologous protein, e.g., an aprotinin, to be expressed in the plant, is placed under the control of a native subgenomic promoter.

RNA plant viruses are suitable for use as expression vectors. The RNA may be single- or double-stranded. Single-stranded RNA viruses preferably may have a plus strand, though a minus strand RNA virus is also intended.

In one embodiment, a recombinant single stranded plus sense plant viral RNA is provided in which a m⁷GpppG₄ cap structure is attached at its 3′ end to an RNA coding sequence for 126-kDa and 183-kDa replicase subunits; said RNA coding sequence for 126-kDa and 183-kDa replicase subunits being attached at its 3′ end to a 30-kDa open reading frame of a viral movement protein, said 30-kDa open reading frame of a viral movement protein containing a coat protein subgenomic promoter sequence, and being attached at its 3′ end to an RNA coding sequence for bovine aprotinin; said RNA coding sequence for bovine aprotinin attached at its 3′ end to a coat protein subgenomic promoter sequence; said coat protein subgenomic promoter sequence attached at its 3′ end to a coat protein coding sequence; said coat protein coding sequence being attached at its 3′ end to a non translatable region; said non translatable region ending in a triple loop structure resembling a tRNA; wherein said RNA coding sequence for 126-kDa and 183-kDa replicase subunits, and said 30-kDa open reading frame of a viral movement protein containing a coat protein subgenomic promoter sequence, are donated by a TMV-U1 strain, and said coat protein subgenomic promoter sequence, said coat protein coding sequence, and said non translatable region ending in a triple loop structure are donated by a TMV-U5 strain.

In another embodiment, a sequence encoding a movement protein is also incorporated into the viral vector because movement proteins promote rapid cell-to-cell movement of the virus in the plant, facilitating systemic infection of the entire plant.

The recombinant viral nucleic acid is prepared by cloning in an appropriate production cell. Conventional cloning techniques (for both DNA and RNA) are well known. For example, with a DNA virus, an origin of replication compatible with the production cell may be spliced to the viral DNA.

With an RNA virus, a full-length DNA copy of the viral genome is first prepared by conventional procedures: for example, the viral RNA is reverse transcribed to form subgenomic pieces of DNA which are rendered double-stranded using DNA polymerases. The DNA is cloned into an appropriate vector and inserted into a production cell. The DNA pieces are mapped and combined in proper sequence to produce a full-length DNA copy of the viral genome. DNA encoding subgenomic promoter sequences with or without a coat protein gene, is inserted into non-essential sites of the viral nucleic acid as described herein. Non-essential sites are those that do not affect the biological properties of the viral nucleic acid or the assembled plant virion. cDNA complementary to the viral RNA is placed under control of a suitable promoter so that (recombinant) viral RNA is produced in the production cell. If the RNA must be capped for infectivity, this is done by conventional techniques. Examples of suitable promoters include the lac, lacuv5, trp, tac, lp1 and ompF promoters. A preferred promoter is the phage SP6 promoter, lambda phage promoter or T₇ RNA polymerase promoter. Production cells can be prokaryotic or eukaryotic and include Escherichia coli, yeast, plant and mammalian cells.

In one embodiment, a recombinant cDNA plasmid is provided which comprises a phage DNA dependent RNA polymerase promoter operably linked to a cDNA sequence encoding a recombinant single stranded plus sense plant viral RNA comprising an RNA coding sequence for 126-kDa and 183-kDa replicase subunits; said RNA coding sequence for 126-kDa and 183-kDa replicase subunits being attached at its 3′ end to a 30-kDa open reading frame of a viral movement protein, said 30-kDa open reading frame of a viral movement protein containing a coat protein subgenomic promoter sequence, and being attached at its 3′ end to an RNA coding sequence for bovine aprotinin; said RNA coding sequence for bovine aprotinin attached at its 3′ end to a coat protein subgenomic promoter sequence; said coat protein subgenomic promoter sequence attached at its 3′ end to a coat protein coding sequence; said coat protein coding sequence being attached at its 3′ end to a non translatable region; said non translatable region ending in a triple loop structure resembling a tRNA; wherein said RNA coding sequence for 126-kDa and 183-kDa replicase subunits, and said 30-kDa open reading frame of a viral movement protein containing a coat protein subgenomic promoter sequence, are donated by a TMV-U1 strain, and said coat protein subgenomic promoter sequence, said coat protein coding sequence, and said non translatable region ending in a triple loop structure are donated by a TMV-U5 strain.

Numerous plant viral vectors are available and well known in the art (Grierson, D. et al. (1984) Plant Molecular Biology, Blackie, London, pp.126-146; Gluzman, Y. et al. (1988 ) Communications in Molecular Biology: Viral Vectors, Cold Spring Harbor Laboratory, New York, pp. 172-189). The viral vector and its control elements must obviously be compatible with the plant host to be infected. Suitable viruses are (a) those from the Tobacco Mosaic virus (TMV) group, such as TMV, Tobacco Mild Green Mosaic virus (TMGMV), Cowpea Mosaic virus (CMV), Alfalfa Mosaic virus (AMV), Cucumber Green Mottle Mosaic virus—watermelon strain (CGMMV-W), Oat Mosaic virus (OMV), (b) viruses from the Brome Mosaic virus (BMV) group, such as BMV, Broad Bean Mottle virus and Cowpea Chlorotic Mottle virus, (c) other viruses such as Rice Necrosis virus (RNV), geminiviruses such as Tomato Golden Mosaic virus (TGMV), Cassava Latent virus (CLV) and Maize Streak virus (MSV).

In a preferred embodiment, vectors based on the plant virus Tobacco Mosaic Virus (TMV) (Pogue et al., Annual Rev. Phytopathol., 40:45-74, 2002), a single stranded RNA genome of approximately 6,400 nucleotides, are used to express r-Aprotinin in plants. Such viral vectors are referred to herein as GENEWARE® vectors. The viral proteins involved in RNA replication are directly transcribed from the genomic RNA, whereas expression of internal genes occurs through the production of subgenomic RNAs. The production of subgenomic RNAs is controlled by RNA sequences in the TMV genome, which function as subgenomic promoters. The CP is translated from a subgenomic RNA and is the most abundant protein and RNA produced in the infected cell. In a TMV-infected plant there are several mg of CP produced per gram of infected tissue. Tobacco mosaic viral expression vectors take advantage of both the strength and duration of this strong subgenomic promoter's activity.

Tobamoviruses have a genomic RNA of approximately 6.4 kb. The genomic RNA is used as an mRNA and translated to produce the replicase protein. TMV produces two replicase proteins, with the larger protein being produced by translational readthrough of an amber (UAG) stop codon. All tobamoviruses produce two smaller coterminal subgenomic RNAs. The coat protein is encoded by the 3′-most RNA, and the movement protein by the larger sgRNA. The virion RNA and sgRNAs are capped. Tobamovirus RNAs are not polyadenylated, but contain a tRNA-like structure at the 3′ end.

Full-length cDNA copies of the TMV RNA genome under the control of the T7 RNA polymerase promoter have been constructed in an E. coli compatible plasmid. Manipulations to the virus cDNA are performed using standard recombinant DNA procedures and the recombinant DNA transcribed in vitro with T7 RNA polymerase to generate infectious RNA. The infectious transcripts are used to infect various tobacco-related species (genus Nicotiana), including tabacum, benthamiana and the proprietary, LSBC-created Nicotiana excelsiana species (Fitzmaurice, U.S. Pat. No. 6,344,597).

GENEWARE® vectors allow expression of the foreign aprotinin protein by adding a heterologous (foreign) coding region (gene) for expression of the mature aprotinin protein preceded by an extensin signal peptide, preferably from N. Benthamiana, in the position of the virus CP coding region so it will be expressed from the endogenous virus coat protein subgenomic promoter. A second gene encoding a CP subgenomic promoter of lesser transcriptional activity and non-identity in sequence is placed downstream of the heterologous aprotinin coding region and a virus CP gene is then added. This encodes a third subgenomic RNA allowing the virus vector to express all requisite genes for virus replication and systemic movement, in addition to the heterologous aprotinin gene intended for overexpression. The cDNA that encodes the resulting recombinant genomic viral vector RNA can be said to contain within it an isolated DNA molecule that comprises a DNA sequence encoding an RNA subgenomic promoter linked at its 3′ end to a DNA sequence that encodes an aprotinin peptide preceded by an extensin signal peptide.

Two (2) events must occur before a functional subgenomic promoter is present. First, the cDNA that encodes the resulting recombinant viral vector must be transcribed in vitro to make infectious plus (+) sense single stranded genomic RNA. The infectious plus (+) sense single stranded RNA must infect a plant host cell and be transcribed to make a complementary minus (−) sense strand. A functional RNA subgenomic promoter capable of recognizing an RNA dependent RNA polymerase is thought either to be present on the (−) strand or result from the presence of both (+) and (−) strands. The RNA subgenomic promoter is said to be operably linked to the (−) sense RNA sequence that encodes the aprotinin peptide. The RNA subgenomic promoter is capable of regulating transcription of subgenomic (−) stranded aprotinin RNA to make its (+) stranded complement. The (+) stranded subgenomic complement is translated to make the peptide that is encoded by the aprotinin coding sequence, and the subsequent plus and minus RNA strand.

These vectors allow expression of r-Aprotinin by adding the sequence for aprotinin and, preferably, for a signal sequence peptide such as extensin, for expression in place of the virus CP so it will be expressed from the endogenous virus coat protein promoter. A second CP promoter of lesser transcriptional activity and non-identity in sequence is placed downstream of the heterologous coding region and a virus CP gene is then added. This encodes a third subgenomic RNA allowing the virus vector to express all requisite genes for virus replication and systemic movement, in addition to the heterologous gene intended for overexpression.

In one embodiment, an isolated DNA molecule is provided and comprises a heterologous aprotinin-encoding DNA sequence; and a promoter-encoding DNA sequence that encodes an RNA subgenomic promoter; wherein said aprotinin-encoding DNA sequence is attached at its 5′ end to said promoter-encoding DNA sequence so that when a resulting negative sense RNA encoded by said promoter-encoding DNA sequence is present, then expression of said aprotinin-encoding DNA sequence is allowed in a plant.

In one embodiment, an isolated DNA molecule is provided which comprises a plus sense single stranded RNA subgenomic promoter sequence, and a plus sense single stranded RNA sequence encoding aprotinin; wherein said sequence encoding aprotinin is linked at its 5′ end to said plus sense single stranded RNA subgenomic promoter sequence.

In one embodiment, a host plant cell transfected with at least one copy of the recombinant single stranded plus sense plant viral RNA is provided, in which the plant viral RNA comprises an RNA coding sequence for 126-kDa and 183-kDa replicase subunits; said RNA coding sequence for 126-kDa and 183-kDa replicase subunits being attached at its 3′ end to a 30-kDa open reading frame of a viral movement protein, said 30-kDa open reading frame of a viral movement protein containing a coat protein subgenomic promoter sequence, and being attached at its 3′ end to an RNA coding sequence for bovine aprotinin; said RNA coding sequence for bovine aprotinin attached at its 3′ end to a coat protein subgenomic promoter sequence; said coat protein subgenomic promoter sequence attached at its 3′ end to a coat protein coding sequence; said coat protein coding sequence being attached at its 3′ end to a non translatable region; said non translatable region ending in a triple loop structure resembling a tRNA; wherein said RNA coding sequence for 126-kDa and 183-kDa replicase subunits, and said 30-kDa open reading frame of a viral movement protein containing a coat protein subgenomic promoter sequence, are donated by a TMV-U1 strain, and said coat protein subgenomic promoter sequence, said coat protein coding sequence, and said non translatable region ending in a triple loop structure are donated by a TMV-U5 strain.

r-Aprotinin is then produced by inoculating plants with a plant viral vector and harvesting the aprotinin transfected plant material. As used herein, the term transfected means that plant material has been infected by the plant viral vector such that the viral vector is being expressed in some part of the plant material. In one embodiment, a virus particle made in a host plant cell by assembly of coat protein subunits around a recombinant single stranded plus sense plant viral RNA is presented. In one embodiment, a host plant is presented which is substantially transfected in its aerial leaves by movement of recombinant single stranded plus sense plant viral RNA, and/or virus particles according from the veins of an infected leaf through the plant to its upper leaves. In one embodiment, a host plant leaf tissue is presented which is transfected by movement of recombinant single stranded plus sense plant viral RNA, and/or virus particles either from one cell to another or by movement through one or more leaf veins.

r-Aprotinin is harvested using filtration and chromatographic methods, as described in the Examples section below. In one embodiment, a method of extracting aprotinin from a transfected host plant or leaf tissue is presented, which includes harvesting leaf or whole plant material; homogenizing the material in the presence of a buffer solution; adjusting the resulting green juice to a pH of about 4; adjusting the temperature of the green juice to between 40 and 50° C.; then cooling the green juice to below 15° C.; filtering the green juice through diatomaceous earth; further filtering through a 0.2 μM depth filter; concentrating the filtrate using a 3 kD MWCO membrane; and diafiltering concentrate with Na phosphate buffer to a conductivity of 3 mS, adjusting the pH of the concentrate to 6.5; filtering the concentrate through a 0.45 μM filter; further separating the filtrate on a SP Sepharose FF resin, collecting the flow through, and eluting the product using a step-gradient containing 20 mM sodium phosphate, and 205 mM NaCl, pH 6.5. The SP Sepharose FF eluent is then filtered through a 0.2 μm filter and pH adjusted to 7.5. Acetonitrile is added to the filtered SP Sepharose FF eluent to a final concentration of 3% ACN and then the SP Sepharose FF eluent is degassed. The degassed SP Sepharose FF eluent is loaded directly onto the 30 micron RPC resin. The flow through is collected, and the product is eluted using a step-gradient containing 20 mM potassium phosphate, 11% ACN, pH 7.5. Reverse phase, eluent fractions are pooled and subjected to concentration using a 1 kD, MWCO membrane and diafiltered against normal saline. The saline diafiltered product is pH-adjusted to 5-7 with HCl or NaOH, and then 0.2 μM sterile-filtered and stored as a pre-sterile bulk at 4-8°. In a method of extracting aprotinin from a plant material using heat and pH treatment, the improvement comprising filtering the green juice through diatomaceous earth or through a ceramic filter.

In one embodiment, aprotinin is extracted from a plant material using cold percolation by passing an amount of water or other solvent through a bed of plant material to form a liquid extract, whereby the liquid extract is recirculated through the bed of plant material for a period of time while maintaining the bed of plant material and recirculating liquid extract at an elevated temperature between room temperature and 60 degrees C., and then recovered as a final liquid extract containing an increased level of active principles, the green juice is filtered through diatomaceous earth.

In one embodiment, aprotinin is extracted from a plant material using a method in which a mixture of at least distilled water and an effective amount of catalyst altered water for improving extraction is at least part of the amount of solvent, and wherein the concentration of the catalyst altered water in the distilled water ranges between 5 and 200 ml of catalyst altered water per 4 liters of distilled water.

In one embodiment, aprotinin is extracted from a plant material using a method in which a the concentration of the catalyst altered water in the distilled water ranges between 5 and 200 ml of catalyst altered water per 4 liters of distilled water.

In one embodiment, aprotinin is extracted from plant tissue or green juice, using a method in which the green juice is filtered through diatomaceous earth. In a preferred embodiment, aprotinin is extracted from plant tissue or green juice, using a method in which the green juice is filtered through a ceramic filter.

In one embodiment, aprotinin is extracted from plant tissue that is subjected to elevated temperature in the presence of a buffer solution, wherein the resulting green juice is cooled to a temperature below 15. degree. C.; and filtered through diatomaceous earth or through a ceramic filter.

In one embodiment, an apparatus is provided for producing aprotinin from plant tissue, in which the plants are harvested prior to grinding the plant tissue into a relatively liquid green juice, whereupon the temperature of the liquid green juice is raised to about 50 degrees C., by means of indirect heat application, followed by subjecting the green juice to a substantially upright hollow cylinder means for housing a bed of diatomaceous earth, and separate inlet and outlet means for said cylinder for flowing a heating medium through said bed of diatomaceous earth.

Another aspect of this invention includes a plant produced recombinant aprotinin variant in which the methionine at position 52 is substituted with an amino acid besides methionine to eliminate oxidation of the methionine. Unlike methionine, the substitute amino acid should not be susceptible to forming sulfone and sulfoxide species. In addition to being less susceptible to oxidation than methionine, a replacement amino acid that will not substantially alter the pI of r-Aprotinin is preferred. Particularly preferred are substitute amino acids for position 52 that will not change the pI of the variant aprotinin by more than a half unit above or below the pI of native aprotinin. In one embodiment, the pI of plant produced aprotinin in which the amino acid at position 52 is an amino acid other than methionine is between about 10 and 11, preferably between about 10.1 and 10.9, more preferably between about 10.2 and 10.8, and more preferably between about 10.4 and 10.6, and most preferably about 10.5.

In one embodiment, the substitute amino acid is leucine, valine, glutamine, glutamic acid or isoleucine. In a preferred embodiment, the substitute amino acid is valine. In a particularly preferred embodiment, the substitute amino acid is glutamine or leucine.

This invention also comprises an aprotinin variant in which the methionine at position 52 is deleted.

Plant expression systems are transformed or transfected with an appropriate plant expression vector encoding an aprotinin variant having a non-methionine amino acid at position 52. In one embodiment, this involves the construction of a transgenic plant by integrating DNA sequences encoding the r-aprotinin variants of the present invention into the plant genome. Methods for such stable transformation are well known in the art.

In a particularly preferred embodiment, viral expression vectors are used to transfect plants through transient infection, as described in detail above and in the Examples section. Both viral and non-viral vectors capable of such transient expression are available (Kumagai, M. H. et al. (1993) Proc. Nat. Acad. Sci. USA 90:427-430; Shivprasad, S. et al. (1999) Virology 255:312-323; Turpen, T. H. et al. (1995) BioTechnology 13:53-57; Pietrzak, M. et al. (1986) Nucleic Acid Res. 14:5857-5868; Hooykaas, P. J. J. and Schilperoort, R. A. (1992) Plant Mol. Biol. 19:15-38). Viral vectors are particularly preferred as they are easier to introduce into host cells and spread through the plant by infection to amplify expression of r-aprotinin variants.

Compositions comprising plant produced aprotinin variants of the present invention are free of microbial and mammalian impurities and contaminants. As used herein, the term impurities refers to components that are part of the host expression system or are intentionally added during the purification process. Contaminants are components that are introduced unintentionally and are not generated by the host system or purification process. Therefore, they are superior to compositions produced by other means, as they do not have impurities that may cause problems for use of aprotinin as a pharmaceutical or as a research tool. The Auerswald patent (U.S. Pat. No. 4,894,436) discloses microbially produced aprotinin variants in which other amino acids are substituted for methionine. However, Auerswald does not disclose a plant produced composition that lacks microbial contaminants. Moreover, it does not address expression in plants or related expression issues and optimization. In addition, the aprotinin variants in Auerswald are produced to allow cyanogen bromide cleavage at one point only in the aprotinin and to avoid such cleavage at position 52. They are not intended to eliminate methionine oxidation at position 52.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

### EXAMPLES

### Example 1

### Cloning of r-Aprotinin

The bovine cDNA sequence (Genbank accession # X05274; Creighton. and Charles. (1987)), covering the coding region of the mature aprotinin protein (amino acid residues #1-58 of SEQ ID NO: 2), was synthesized and fused to the coding region for a plant signal peptide sequence derived from the N. benthamiana gene (amino acids #-1-26 of SEQ ID NO: 1) that has homology to the N. plumbaginifolia extensin gene (Genbank accession # M34371; De Loose et al (1991)). This chimeric gene was cloned into the TMV-based expression vector DN5 via Pacd and XhoI cloning sites to generate the plasmid pLSB2602, set forth in SEQ ID NO: 3. The DN5 vector is a recombinant vector containing most of the TMV genome (U1 strain replicase and movement proteins) and part of the tobacco mild green mosaic virus genome (TMGMV; U5 strain coat protein and 3′ nontranslated region) in a pUC plasmid. This arrangement of U1 and U5 sequences provides an extra subgenomic promoter for expression of the aprotinin gene. The replacement of the U1 coat protein by the U5 coat protein keeps rearrangement to a minimum resulting in a more stable expression vector.

The presence of the aprotinin gene insert was confirmed by sequence analysis and by restriction enzyme mapping of the pLSB2602 plasmid. The intact nature of the aprotinin gene was also confirmed by restriction enzyme mapping (PacI/XhoI to liberate the intact extensin-aprotinin insert; NcoI/PstI to confirm the presence of a restriction site within the aprotinin insert and a site within the virus expression vector). The nucleotide and translated amino acid sequences of the insert (extensin-Aprotinin) are shown in SEQ ID NO: 1. The mature aprotinin peptide is 58 amino acids long and its molecular weight is 6512 Daltons.

Method for Subcloning Aprotinin Gene Sequence into Vector

Plasmid pLSB2602 contains the mature bovine aprotinin-coding region with the N. benthamiana extensin signal peptide. Plasmid DNA was isolated from a transformed E. coli culture. The isolated DNA was digested with HindIII to check for the intactness of the plasmid and NcoI/PstI to check for the presence of the aprotinin gene. Furthermore, the complete sequence of the plasmid was determined in order to identify the DN5 vector backbone and to confirm that there were no mutations in the aprotinin gene.

Plasmid pLSB2602, containing the aprotinin gene, is transcribed in vitro using T7 RNA polymerase and transcripts are inoculated on phloem source leaves present on 21 day post sowing N. benthamiana plants. Characteristic viral symptoms, vein clearing and leaf curling, are noted ˜6-14 days post inoculation. Interstitial fluids (IF) were isolated from these plants and analyzed by SDS-PAGE for the presence of aprotinin. The identity and activity of aprotinin in the interstitial fluid (IF) were determined by MALDI-TOF and by trypsin inhibition assay, respectively.

Detailed Method for Subdloning Aprotinin Gene Sequence into Vector
1. Synthesize aprotinin gene sequence to be subcloned into full-length tobamovirus vector plasmid, containing a portion of the TMV genome. Overlapping oligonucleotides with flanking cohesive ends are chemically synthesized for ligation into prepared vectors. Directional cloning is used. The plasmids bearing the virus genomes are digested with appropriate restriction endonucleases to completion and purified from agarose gels prior to ligation.
2. Ligate the aprotinin gene sequence into vector plasmid using a >5-fold excess insert:vector ratios.
3. Transform ligations into highly competent E. coli strains and prepare plasmid DNA using standard methods. Commonly used strains of E. coli are JM109 and DH5α. Expected plasmid yield from 50 ml cultures using standard LB with ampicillin (50-100 μg/ml) is between 50 and 100 μg of recombinant plasmid following purification by standard methods. It is important to test the infectivity and expression characteristics of virus transcripts derived from 2-3 plasmid clones representing the same construct.

In vitro transcription of TMV vectors
1. Linearize 1-10 μg of plasmid with endonuclease (50 μl final volume) according to manufacturer's instructions. Not necessary with pBSG 1057 Rbz due to 3′ ribozyme, but for maximal infectivity of the 30B Rbz transcripts, linearization with Kpn I or Pst I can be done.
2. Extract restriction enzyme digested DNA 2× with equal volumes of phenol:chloroform:iso-amyl alcohol (25:24:1) to remove contaminating nucleases.
3. Precipitate linearized DNA template with addition of 0.33 volume (vol.) 10M ammonium acetate (NH4bAc) and 2.5 vol. of 100% ethanol (EtOH). Place on dry ice 15 minutes, spin at 15,000×g for 10-15 minutes to pellet DNA. Wash pellet with 70% EtOH. Dry in speed vacuum centrifuge. Resuspend pellet in nuclease-free dH₂O at a final DNA concentration of 0.5 μg/μl.
4. Transcribe DNA template with phage T7 polymerase.
   2.5 μl 10× transcription buffer (New England Biolabs, NEB)
   0.5 μl 40U/μl RNasin (Promega)
   1.25 μl 20 mM rATP, rCTP, rUTP(each)/2 mM RGTP
   1.25 μl 5 mM GpppG or 7MGpppG cap analog (NEB)
   1 to 2 μg linearized template DNA
   1 μl Phage RNA polymerase (T7, NEB)
   Adjust volume to 25 μl with RNase free sterile distilled water (dH₂O).
   Mix all reaction components with gentle pipetting.
Incubate at 37° C. for 1 hour.
   5. Analyze transcripts by agarose gel electrophoresis.
      With addition of FES buffer (100 μl; 0.1 M glycine, 0.06 M K₂HPO₄ buffer containing 1% sodium pyrophosphate, 1% macaloid, 1% celite; pH to 8.5-9.0 with phosphoric acid), approximately 3-8 plants can be inoculated with the RNA products.

Inoculation of “Inoculum” plants with TMV vector transcripts:
1. Add 100 μl FES transcript inoculation buffer to the remaining 23 μl of transcription reaction. Place on wet ice when not in use.
2. Place 10 drops of inoculum every 1-3 cm along the leaves of Nicotiana benthamiana (a total of about 10-20 μl per leaf). Gently spread the inoculum over the leaf with a sterile cotton swab or by finger with a rubber lab glove.
3. Place plants under appropriate growing conditions. Systemic symptoms appear in 1-2 weeks.

Virus purification for Large Scale Inoculum
1. Homogenize frozen, systemically infected leaves in virus extraction buffer (250 mM NaCl, 0.264% iso-ascorbic acid, 0.1% sodium metabisulfite, 5 mM EDTA, pH 4.0; 1 ml extraction buffer per gm infected tissue). Adjust pH of homogenate to 4.0. Frozen tissue should be at −80° C. in plastic bag and crumbled while still frozen and then poured into blender. Tissue should be homogenized for approximately two minutes on high in a Waring blender.
2. Centrifuge for 10 minutes at 10,000×g. Decant and save the supernatant.
3. The supernatant fraction is saved for sodium dodecyl sulfate-polyacrylamide gel electrophoresis (SDS-PAGE analysis) and activity analyses.

### Example 2

### Characterization of Plant-Produced r-Aprotinin

Representative plants are extracted at 14 days post inoculation by a homogenization method and analyzed for the presence of aprotinin protein and activity. Leaves are weighed and ground in the extraction buffer (250 mM NaCl, 0.264% iso-ascorbic acid, 0.1% sodium metabisulfite, 5 mM EDTA, pH 4). The pH of this homogenate is adjusted to 4.0 and clarified by centriftigation at 10,000×g for 10 minutes. The supernatant fraction is saved for sodium dodecyl sulfate-polyacrylamide gel electrophoresis (SDS-PAGE analysis) and activity analyses.

The supernatant fraction is also analyzed for its ability to inhibit the proteolytic activity of porcine trypsin (Fritz et al, 1966; Kassell, 1970). The inhibitory effect of aprotinin on porcine trypsin is determined by monitoring the release of p-nitroaniline from the substrate N-a-benzoyl-L-arginine-p-nitranilide (BAPA). One trypsin inhibitory unit (IU) of BAPA is defined as the amount of inhibitor that reduces the activity of two trypsin units by 50%. The activity of the supernatant fraction is typically >1.25 IU (or 769 Kallikrein Inactivation Unit (KIU); 1 IU BAPA=615 KIU) Extracts isolated from non-inoculated plants do not exhibit any detectable inhibition of trypsin activity (data not shown).

Table 1 summarizes these assays and their acceptance criteria.

TABLE 1
Acceptance Criteria for pLSB2602

| Assay | Acceptance Criteria |
|---|---|
| Appearance | Clear, colorless, and has no floating particulate |
| DNA concentration | 0.5 to 7.0 mg/mL |
| Restriction pattern | The HindIII and NcoI/PstI restriction |
| | pattern must match the reference |
| | pLSB2602 and the predicted patterns. |
| DNA sequence | No mutation in the aprotinin gene. |
| | The vector sequence matches the predicted |
| | sequence of the DN5 backbone |
| Expression in plants | 1. Visible aprotinin band by SDS-PAGE |
| | 2. The molecular weight is 6512 +/− 3.26 |
| | by MALDI-TOF |
| | 3. The aprotinin concentration is ≧0.01 mg/ml |
| | by activity assay. |

Once these criteria were met, the pLSB2602 was aliquoted into 300 1.5 mL polypropylene tubes each containing 2 μL of DNA at 10 ng/μL concentration and placed in −70° C. freezers.

### Example 3

### Scale-Up of Production and Purification of r-Aprotinin.

Nicotiana seeds lots, including those from Nicotiana excelsiana, are generated from plants that are grown to maturity in a clean, isolated environment. The seed is characterized based on the morphology of a mature plant, susceptibility to TMV-based vector infection as well as yield and quality of a standardized r-Aprotinin product.

The characterized Nicotiana seeds are used to propagate plants for r-Aprotinin production. These seed lots are coated with a clay-based pellet to increase individual seed size and improve handling during the seeding process. Pelletized seed is tested regularly for germination rate.

Standard agronomic practices are utilized for field production. Multiple disk harrowing and cultivation passes are used to prepare the field for transplanting. Fertilizers are applied according to recommendations provide by soil test analysis. Pre-planting soil-applied treatments are applied according to the labeled rates.

Seedlings are mechanically transplanted at the field site. Each plant is spaced 14 inches apart within the row. Six, 19-inch rows are separated by a 72-inch “skip-row” to facilitate equipment movement within the filed site. Seedlings are transplanted at a density of approximately 14,500 plants per acre. After transplantation, fields are monitored for general field appearance, plant height and vigor, and TMV symptomatology (post inoculation).

Each field site is regulated under an issued USDA/APHIS/BRS release permit that is applied for or renewed each year (APHIS 2000 permit application). The permit and supplemental permit conditions designate the regulated article containment parameters and protocols that ensure TMV and product containment. USDA/APHIS/BRS conducts routine inspections of the field sites to ensure compliance.

Inoculum Preparation and Plant Inoculation

Infectious in-vitro transcripts are synthesized from pLSB2602, Master or Working Plasmid Bank DNA, containing the aprotinin gene, and are used to inoculate N. benthamiana plants. N. bethamiana is used as a vector packaging intermediate host plant that supports the rapid accumulation of TMV virions. Virions isolated from these inoculated plants, at 6-8 days post inoculation, are assayed for aprotinin expression in Nicotiana plants, including Nicotiana excelsiana, and for aprotinin gene insert integrity. Plants inoculated with the virion inoculum are tested for the presence of expressed aprotinin and for the quantity of TMV coat protein expressed. Potential aprotinin gene anomalies that may be present in the virion preparation are evaluated by reverse transcriptase-polymerase chain reaction (RT-PCR). RNA is extracted from the virion preparation and is subjected to first strand cDNA synthesis. Subsequently, PCR is performed on the cDNA as templates using oligonucleotides (5696S and 5851A) flanking the aprotinin insert. Any deletions occurring in the aprotinin gene would be detected by analysis of the products on an agarose gel. The predicted full-length RT-PCR product is 420 bp. Similar to the Master Plasmid Bank, the inoculum is evaluated for the expression of aprotinin in Nicotiana. Table 2 shows the release specifications for the inoculum. The inoculum is titered by local lesion assay on the upper leaves of a 27-37 days post sowing, N. tabacum cv Xanthi NN plant. The virions (inoculum) are aliquoted into 17 ml, 5 ml, and 1 ml fractions and stored in two separate −20° C. freezers until required for preparation of the inoculum for plant inoculation.

TABLE 2
The Release Specification of the Inoculum
for aprotinin manufacture.

| Assay | Release Specifications |
|---|---|
| Virion concentration | Quantify the amount of coat protein |
| | by SDS-PAGE gel densitometry |
| RT-PCR | Only full-length RT-PCR product (420 bp) |
| | presence and the absence of any |
| | deleted products. |
| Expression in plants | 1. Visible aprotinin band by a SDS-PAGE |
| using virions as | 2. The molecular weight is 6,512 Da ± 0.05% |
| inoculum | by MALDI-TOF |
| | 3. The aprotinin concentration is ≧0.01 mg/mL |
| | by activity assay. |

The quantity of the r-Aprotinin virion to be inoculated on each plant is determined using SDS-PAGE analysis of TMV coat protein. For field production, the target application rate is 1 μg of TMV coat protein per plant. The volume of the r-Aprotinin virion that is used for each inoculum batch is calculated using the results of the SDS-PAGE analysis.

The inoculum used for field production is mixed in approximately 16 L batches. Each batch contains a 0.5 M NaKPO₄ buffer (2% by volume), diatomaceous earth (1% by volume), the r-aprotinin viral vector, and deionized water. The r-Aprotinin virion is thawed just prior to addition to the inoculum solution. The prepared inoculum is agitated during the inoculation process. The inoculum is drawn through Tygon tubing attached to an air gun. Each plant is sprayed with approximately 2.0 mL of inoculum solution propelled by about 100 psi air pressure. Dependent upon growth conditions, plants are inoculated 2-4 weeks post transplanting. At the completion of the inoculation process, all equipment is cleaned with a 0.625% sodium hypochlorite solution and rinsed thoroughly with fresh water.

Planat Harvest, Extraction, Downstream Purification and Bulk Hold

The r-Aprotinin protein is accumulated in the leaf and is recovered from the leaf material by a process of tissue homogenization and “green juice” clarification. Through a series of chromatography steps which include SP Sepharose chromatography and reverse phase HPLC and ultrafiltration, the r-Aprotinin product is separated from host components.

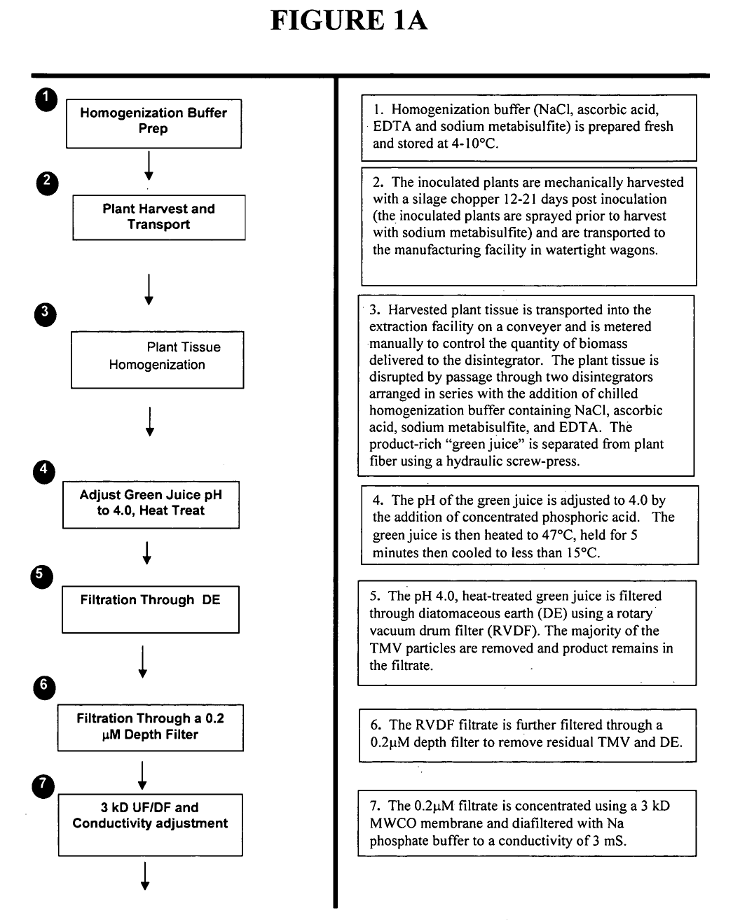

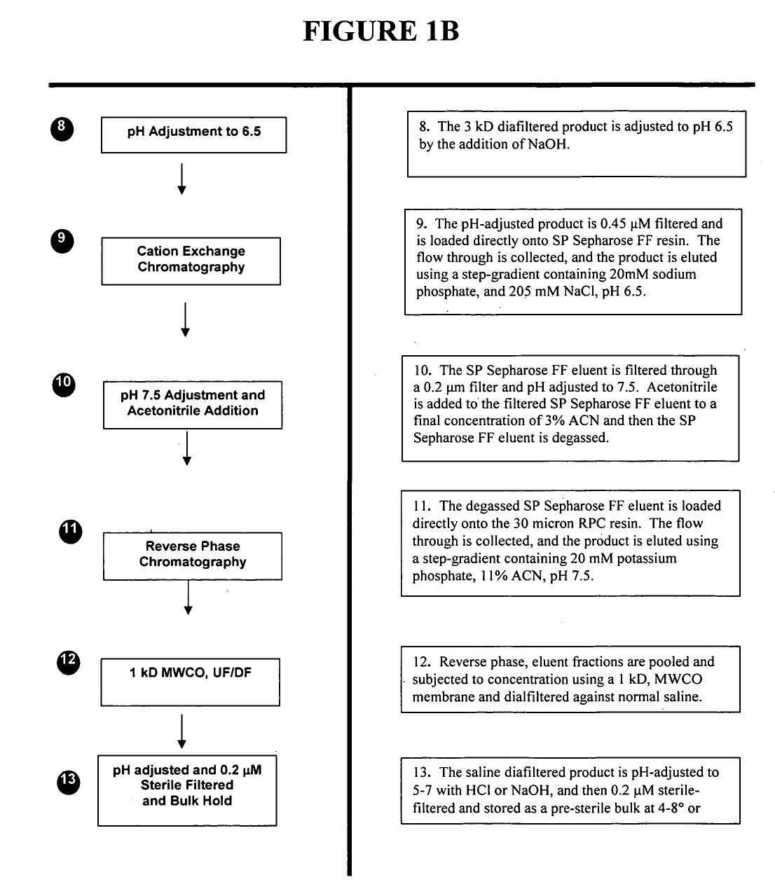

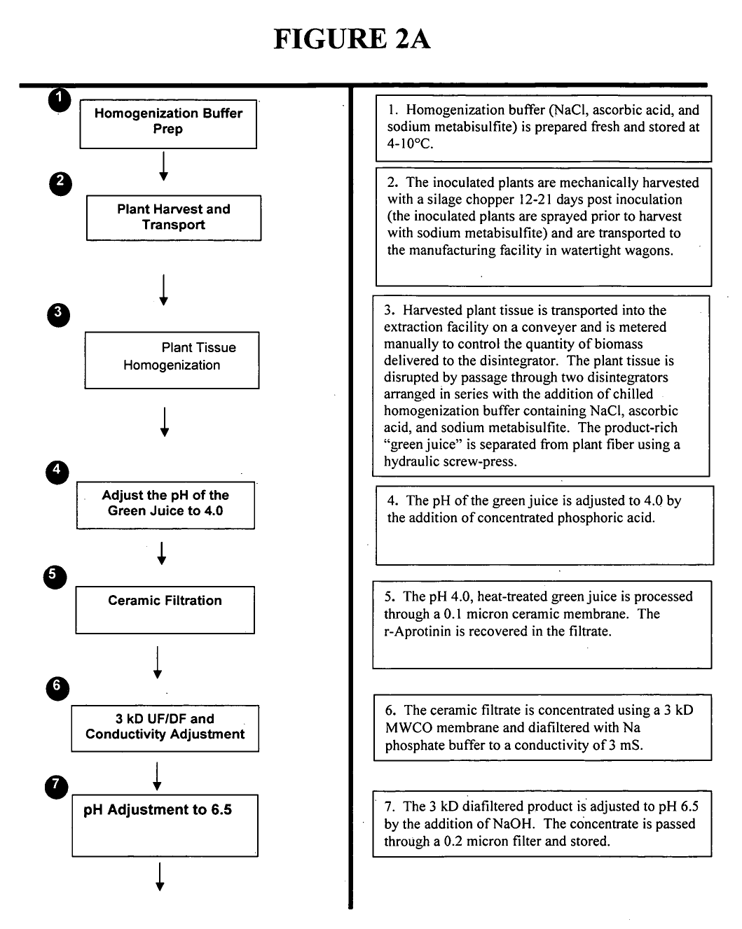

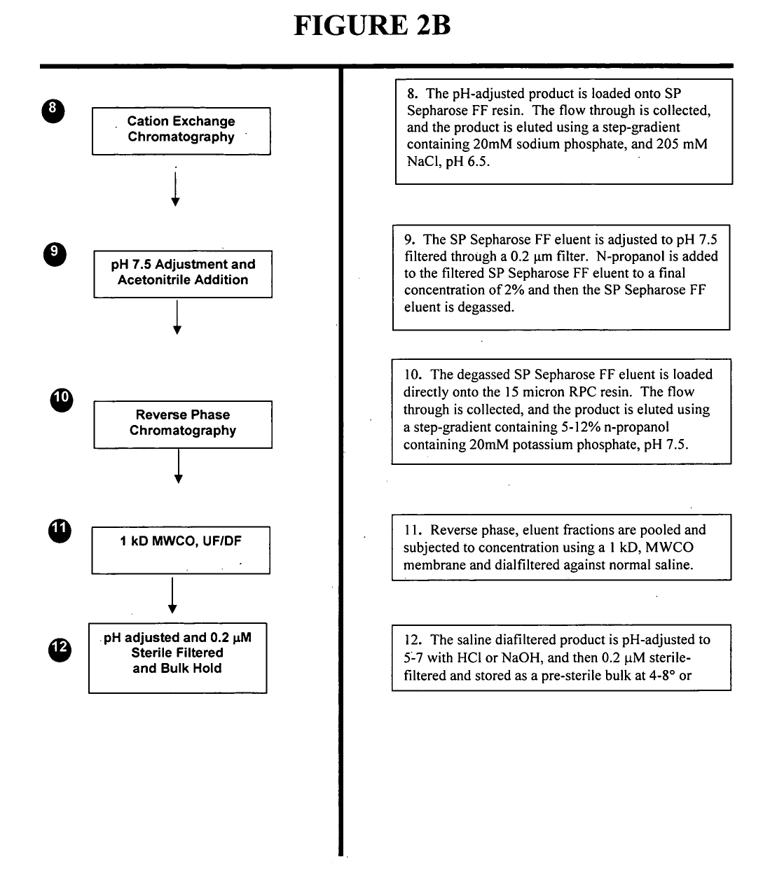

A schematic flow diagram of the process used to purify the r-Aprotinin protein is presented in FIGS. 1 and 2 and is described below.

Homogenization Buffer Preparation

Homogenization buffer, containing NaCl, ascorbic acid and sodium metabisulfite, is prepared in advance and stored at 4-10° C. to minimize potential microbial growth. Homogenization buffer components have been selected to inhibit oxidation and polyphenol formation.

Plant Harvest and Transport

Aprotinin-containing plants are harvested 12 to 21 days post inoculation. The vector-infected plants are sprayed with sodium metabisulfite prior to harvesting to inhibit microbial growth. Plants are mechanically harvested with a silage chopper and are directly loaded into watertight wagons for transport to the manufacturing facility. Alternatively, plants grown and inoculated at a site remote to the manufacturing facility are harvested, chilled to 4-10° C., placed in a USDA approved shipping container and transported to the manufacturing facility for processing within 48 hr of harvest.

Plant Tissue Homogenization

The plant tissue is delivered inside the manufacturing facility by a conveyor system and is manually metered to a dual disintegration system comprised of knives, hammers and screens. The pulverized plant material enters a horizontal hydraulic screw press that uses an auger and screen to separate the fiber (waste) from the product-containing green juice. Homogenization buffer is added at both the primary grinder and the press to aid in the extraction of product.

Green Juice Clarification and Product Concentration

Green juice is pumped from the press to a stirred tank where the pH is adjusted to 4.0 with the addition of phosphoric acid. The pH adjustment occurs in a dynamic system that is controlled by Programmable Logic Control (PLC). The pH 4.0-adjusted green juice is processed through a 0.1 micron ceramic membrane (Pall Life Sciences, East Hills, N.Y.). The r-Aprotinin is recovered in the filtrate and concentrated using a 3 kD MWCO membrane and diafiltered with sodium phosphate buffer to a conductivity of 3 mS. The UF/DF product is adjusted to a pH of 6.5 by the addition of NaOH. In another embodiment, the green juice may also be pumped into the drum of a Rotary Vacuum Drum Filter where under vacuum, the green juice is clarified by filtration through diatomaceous earth (DE). The filtrate is then filtered through a minimal micron depth filter to further clarify and remove residual DE. Filtrate is then concentrated as above. The method using DE has several disadvantages compared to the method using the ceramic membrane.

SP Sepharose FF Chromatography

The pH 6.5-adjusted concentrate obtained using the ceramic filter is loaded directly onto a column containing SP Sepharose FF resin equilibrated with 20 mM sodium phosphate, pH 6.5 at room temperature. SP Sepharose FF is a strong cation exchanger that enables the capture and purification of the product. Unbound proteins are washed from the resin first with the equilibration buffer until the Iw returns to baseline, then followed with 20 mM sodium phosphate, 130 mM NaCl, pH 6.5, and finally with 20 mM sodium phosphate, 180 mM NaCl, pH 6.5 washes. Aprotinin is eluted from the resin using a step-gradient of 20 mM sodium phosphate, 205 mM NaCl, pH 6.5.

SP Sepharose FF Eluent Filtration

The eluent from the SP Sepharose FF column is filtered through a 0.2 μm capsule filter. N-propanol is added to the SP Sepharose FF eluent to a final concentration of 2%. Acetonitrile may also be used to a final concentration of 3%. The SP Sepharose FF eluent is degassed before loading onto the RPC resin.

30 μm Reverse Phase Chromatography

The degassed SP Sepharose FF eluent is loaded directly onto a column containing 30 μm RPC equilibrated with 20 mM potassium phosphate, 3% acetonitrile, pH 7.5. Unbound proteins are washed from the resin with the equilibration buffer until the UV signal returns to baseline. Aprotinin is eluted using a step gradient of 20 mM potassium phosphate, 11% acetonitrile, pH 7.5. The chromatography is carried out at room temperature. In another embodiment, the column contains 15 μm RPC equilibrated with 20 mM potassium phosphate, 2% n-propoanol, pH 7.5 and product is eluted using a linear gradient from 5 to 12% n-propanol containing 20 mM potassium phosphate, pH 7.5.

Ultrafiltration and Diafiltration

The reverse phase eluent is concentrated using an ultrafiltration system containing a 1 kD MWCO, stabilized cellulose membrane. The reverse phase eluent is concentrated to 10 mg/mL by ultrafiltration and then diafiltered against 5-7 volumes of chilled (4-8° C.) saline. Diafiltration is performed by matching the influx of the diafiltration buffer to the flux rate of permeate production.

pH Adjustment, 0.2 μm Sterile Filtration and Bulk Hold

The concentrated r-Aprotinin is pH-adjusted to 5-7 with HCl or NaOH, and then 0.2 μm sterile filtered into either glass or polypropylene containers and stored at 4-8° C. or −20° C. for a period up to 6 months prior to finish and fill operations.

Release testing of the plant-expressed r-Aprotinin is performed using selected methods described in detail below.

Description of Test Methods

SDS-PAGE

All r-Aprotinin API lots are analyzed by 16% Tris-glycine SDS-PAGE (LSBC SOP# QCA-106). After electrophoresis, gels are stained with Coomassie Brilliant Blue, placed on a light box and scanned with a BioRad densitometer. Images are stored on disk. Relative quantities of the r-Aprotinin API are determined by densitometry using BioRad Software.

Reversed-Phase High Performance Liquid Chromatography (RP-HPLC)

The purity of the final container lots of r-Aprotinin API are determined by RP-HPLC to quantitatively determine impurities in the product with identification by MALDI-TOF MS.

Appearance

The appearance of r-Aprotinin API in solution is determined by visual inspection. This method assures that the final product is a clear, colorless solution that is free of visible particulates.

pH Measurement

The pH of the r-Aprotinin API filled in the final container is determined by standard methods using a calibrated pH meter.

Protein Concentration

The protein concentration is determined for each lot of r-Aprotinin API using the OD280 method or by BCA (Pierce Laboratories) using Trasylol® as a standard.

Specific Activity

The specific activity of r-Aprotinin API lots is determined as Kallikrein Inactivation Units (KIU) or trypsin inhibition units (TIU). One trypsin inhibitor unit (TIUBAPA) will decrease the activity of two trypsin units by 50% where one trypsin unit (TUBAPA) will hydrolyze 1.0 micromole of Na-benzoyl-L-arginine-p-nitroanilide (L-BAPA) per minute at pH 7.8, 25° C., determined photometrically at 405 nm. The biological unit of Kallikrein Inactivation (KMU) of r-Aprotinin API was calculated from TIUBAPA (Fritz and Wunderer, 1983).

Residual DNA

The r-Aprotinin API filled in the final container is tested for the presence of residual plant-derived nucleic acids using a DNA ³³P probe hybridization technique. DNA probes derived from plant genomic DNA are used in this method. This method has a lower limit of detection of 10 μg DNA.

Sterility

The direct inoculation method for determination of r-Aprotinin API lot sterility is performed according to 21 CFR 610.12, by Northview Laboratories.

Limulus Amebocyte Lysate Test (LAL)

LAL testing is performed on r-Aprotinin in the final container by Northview Laboratories for endotoxin levels, using a validated method for this material.

Selected Organic Molecules

The final container lots of r-Aprotinin API are screened for the presence of low molecular weight impurities (i.e., nicotine and acetonitrile) that may be present using Gas. Chromatography/Mass Spectrometry.

Local Lesion Host Assay

The final container lots of r-Aprotinin API are tested for recombinant TMV infectivity by the local lesion host assay. The presence of infectious TMV is visualized by the formation of plaques or necrotic lesions formed on host plant leaves.

Trace Metal Toxicants Analysis

The presence of heavy metals will be evaluated during product development to ensure that the final r-Aprotinin API product meets specifications. Nicotiana plants are not known to accumulate heavy metals and all field sites that are used to produce r-Aprotinin are selected based upon a history of food/feed crop production, i.e. soils known to be free of heavy metal contamination. Heavy metal analysis is performed by Irvine Analytical Laboratory, Irvine, Calif., using validated methods.

Pesticides Analysis

No pesticides, herbicides or fungicides were used during growth of plants for the r-Aprotinin API Lot #040413LYF, therefore no analyses for these compounds were performed on this lot. Only pesticides registered and approved by the Environmental Protection Agency may be used if needed, and if used, API and in-process samples will be analyzed for pesticide residues using validated methods.

2.4 Comparison of r-Aprotinin to Trasylol®

Plant-expressed r-Aprotinin API was tested using the identity, purity and potency assays described above and in Table 4 and the results were compared with those obtained from Trasylol®.

The results of these biochemical analyses indicate that plant-derived r-Aprotinin is comparable to Trasylol® with respect to identity, purity and potency.

TABLE 4
Test Methods and Results of Aprotinin Comparisons

| | Comparative | | |
|---|---|---|---|
| Assay | Attribute | r-Aprotinin | Trasylol ® |
| Identity by | Conforms with | Conforms | Conforms |
| Tryptic Digest | bovine lung | | |
| MALDI-TOF | aprotinin pre- | | |
| mass mapping | dicted tryptic | | |
| | fragments and | | |
| | fragment deri- | | |
| | vatives (84% | | |
| | amino acid | | |
| | coverage) | | |
| Identity by | 6,512 Da ± 0.05% | 6,512 Da | 6,512 Da |
| MALDI-TOF MS | | | |
| Identity by | Conforms with | Conforms | Conforms |
| Amino Acid | bovine lung | | |
| Analysis | aprotinin amino | | |
| | acid composition | | |
| Purity by | Purity | >99% | >99% |
| SDS-PAGE | | | |
| Purity by | Purity | 87.6% + | 86.3% + |
| RP-HPLC | | 12.4(Ox) % | 5.7(Ox) % |
| Purity by | Purity | Comparable | Comparable |
| GC/MS | | levels of | levels of |
| Small molecular | | target | target |
| weight host | | compounds | compounds |
| toxicants | | | |
| Purity by | Clear, colorless, | Clear, | Clear, |
| Appearance | free of visible | colorless, | colorless, |
| | particles | particle free | particle free |
| Potency by | >6,500 KIU/mg | 7,175 KIU | 6,859 KIU |
| Specific | protein | | |
| Activity | | | |

Amino Acid Analysis (AAA)

The theoretical amino acid composition for Aprotinin is shown in the Table 5 with results for r-Aprotinin, Lot# 040413LYF and Trasylol®. Identical results were obtained for the two Aprotinin lots.

TABLE 5
Amino Acid Analysis of r-Aprotinin and Trasylol ®

| | | Theoretical | r-Aprotinin | Trasylol ® |
|---|---|---|---|---|
| | | (Sequence) | Lot # 040413LYF | Lot # 25008RG |
| Res | % | # | # | # |
| Asp | 3.45 | 2 | 5* | 5* |
| Asn | 5.17 | 3 | 0 | 0 |
| Thr | 5.17 | 3 | 3 | 3 |
| Ser | 1.72 | 1 | 1 | 1 |
| Glu | 3.45 | 2 | 3* | 3* |
| Gln | 1.72 | 1 | 0 | 0 |
| Pro | 6.90 | 4 | 5** | 5** |
| Gly | 10.34 | 6 | 6 | 6 |
| Ala | 10.34 | 6 | 6 | 6 |
| Val | 1.72 | 1 | 1 | 1 |
| Cys | 10.34 | 6 | ** | ** |
| Met | 1.72 | 1 | 1 | 1 |
| Ile | 3.45 | 2 | 2 | 2 |
| Leu | 3.45 | 2 | 2 | 2 |
| Tyr | 6.90 | 4 | 4 | 4 |
| Phe | 6.90 | 4 | 4 | 4 |
| Lys | 6.90 | 4 | 4 | 4 |
| His | 0.00 | 0 | 0 | 0 |
| Trp | 0.00 | 0 | 0 | 0 |
| Arg | 10.34 | 6 | 7 | 7 |

*Glutamine and asparagine are converted to glutamic acid and aspartic acid, respectively, during the analytical process. There was one glutamine and three asparagines which were converted yielding 5 aspartic acids and 3 glutamic acids, respectively.
**Cysteine co-elutes with proline, therefore could not be determined for either product.

Purity by SDS-PA GE

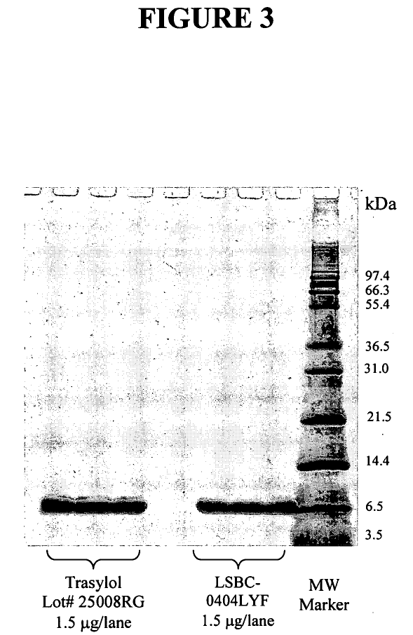

An example of the purity results for Aprotinin is shown in FIG. 3. The three lanes on the left of this gel contain the Trasylol® Aprotinin and the three lanes next to the molecular weight markers contain Applicants' r-Aprotinin API from the lot indicated. At a protein load of 1.5 μg per lane, no Coomassie-stained band other than Aprotinin is detected in either Aprotinin sample.

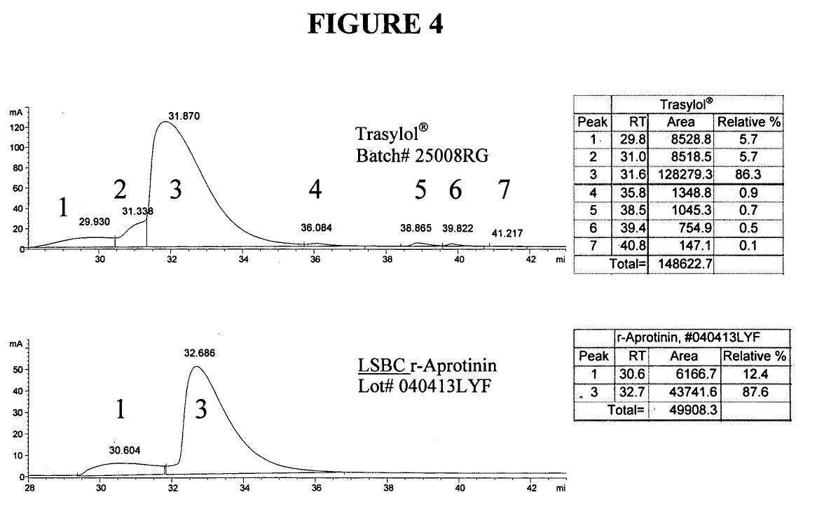

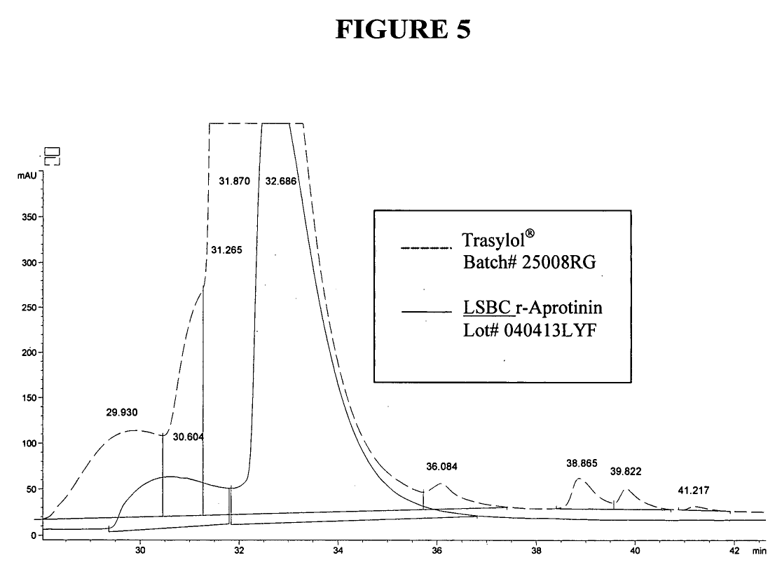

RP-HPLC analysis was performed on r-Aprotinin, lot# 040413LYF, and on Trasylol®. The resulting chromatograms are shown in FIG. 4, with relative peak areas given in the tables to the right of corresponding Figures. At least seven peaks were detected in the Trasylol® chromatogram representing at least 11 different components, compared to only two being detected in the r-Aprotinin lot# 040413LYF sample. An overlay of the chromatograms is shown in FIG. 5. Each peak was collected and subjected to MALDI-TOF MS and proteins were identified by tryptic-MALDI analysis. The two peaks (3 and 1) in the r-Aprotinin, Lot # 040413LYF, sample were identified as r-Aprotinin (87.6%) and oxidized r-Aprotinin (12.4%), respectively. No other quantifiable impurities were detected in the r-Aprotinin sample. Trasylol® contained seven peaks that were identified as Aprotinin (86.3%), oxidized Aprotinin (5.7%), two C-terminal truncated Aprotinin (-alanine and -glycine-alanine) accounting for 5.7% and what appears to be acetylated and various other derivatized Aprotinin species accounting for the other 2.3%. Also, oxidized forms of the truncated species were detected in the Trasylol® sample.

Purity by GC/MS

Several small molecular weight impurities (plant metabolites) were determined in the final product of r-Aprotinin (lot# 040413LYF) and the Trasylol® samples by GC/MS as described in SOP# QCA-109. Compounds monitored included, nicotine, (a major metabolite in Nicotiana plants) and acetonitrile (a process eluant). Compounds detected in the plant-produced r-Aprotinin are shown in Table 6 below. In addition, the same compounds were monitored and detected in the Trasylolo sample. The results are listed for comparison purposes. Based on LD₅₀ values for each compound, the measured concentrations do not represent a safety hazard for the expected dosages for this product. The total amount of each compound, based on a 700 mg aprotinin dose, is less than what would be obtained from the smoke of a single cigarette.

TABLE 6
Small Molecular Weight Impurities

| | | | μg Target Compound | |
|---|---|---|---|---|
| | | | per 700 mg Aprotinin* | |
| | In Rats | | r-Aprotinin | Trasylol ® |
| Compound | LD₅₀ | | Lot #040413LYF | Lot # 25008RG |
| Toluene | 7.5 | g/kg | 17.6 | 8.8 |
| Acetonitrile | 3.8 | g/kg | ND** | ND** |
| 2-Furfural | 127 | mg/kg | 19.7 | 6.8 |
| m/p-Cresol | 2.02/1.8 | g/kg | 28.7 | 10.0 |
| Nicotine | 0.3 | (i.v.) | ND** | ND** |
| (in mice) | 9.5 | (i/p.) mg/kg | | |

*Each value represents the mean for three analyses.
**ND - Below the limit of detection.

Comments on Oxidized Species and Their Control

Both the Trasylol® product and r-Aprotinin contain oxidized species of the Aprotinin. Oxidation of the API in Trasylolo and in r-Aprotinin occurs at the identical methionine residue at position 52 on the molecule. Both single-O (+16 MW) and doublet-O (+32 MW) oxidation species are detected in both products.

### Example 4

### Cloning, Production and Characterization of Aprotinin Variants

The method selected to reduce or eliminate r-Aprotinin methionine oxidation involves the removal of the methionine residue or substituting the methionine residue with a different amino acid that is less susceptible to oxidation. Substitute amino acids were chosen such that the pI of the side chain and pKs of the amino and carboxylic group were as close as possible to those of Methionine. Preferred amino acids do not substantially affect the pI of native aprotinin and, therefore, do not substantially alter processing and purification. Leu, Ile and Val have slightly higher molar absorbance (about 0.002 to 0.004) and Glutamine will not change the molar absorbance. Expected changes in pI, molar absorbance and mass of mutant compared to native form with Met at position 52 follow.

Name: Native Processed Aprotinin

Average mass (Da): 6511.5315
Monoisotopic mass (Da): 6507.0414
Molar ext. coeff. (280nm): 5480
Molar absorbance (280nm): 0.842
Theoretical pI (SS/SH): (1) 10.53/9.02—(2) 10.62/9.02—(3) 10.51/0.00
   Name: L mut Aprotinin
Average mass (Da): 6493.4924
Monoisotopic mass (Da): 6489.0850
Molar ext. coeff. (280 nm): 5480
Molar absorbance (280 nm): 0.844
Theoretical p1 (SS/SH): (1) 10.53/9.02—(2) 10.62/9.02—(3) 10.51/0.00 Number of residues: 58
   Name: Ile mut Aprotinin
Average mass (Da): 6493.4924
Monoisotopic mass (Da): 6489.0850
Molar ext. coeff. (280 nm): 5480
Molar absorbance (280 nm): 0.844
Theoretical pI (SS/SH): (1) 10.53/9.02—(2) 10.62/9.02—(3) 10.51/0.00 Number of residues: 58
   Name: V mut Aprotinin
Average mass (Da): 6479.4655
Monoisotopic mass (Da): 6475.0693
Molar ext. coeff. (280 nm): 5480
Molar absorbance (280 nm): 0.846
Theoretical pI (SS/SH): (1) 10.53/9.02—(2) 10.62/9.02—(3) 10.51/0.00
   Name: Q mut Aprotinin (Gln)
Average mass (Da): 6508.4636
Monoisotopic mass (Da): 6504.0595
Molar ext. coeff. (280 nm): 5480
Molar absorbance (280 nm): 0.842
Theoretical pI (SS/SH): (1) 10.53/9.02—(2) 10.62/9.02—(3) 10.51/0.00

Based on the above considerations, four Geneware® r-Aprotinin methionine analogs were produced as follows:
1) Removal of the methionine residue (Mutant M)
2) Substitution of the methionine with valine (Mutant V)
3) Substitution of the methionine with leucine (Mutant L)
4) Substitution of the methionine with glutamine (Mutant Q)

Plant viral expression vectors containing each of the mutations were created using pLSB2602 as a starting point. Polymerase chain reaction with appropriate oligonucleotides was used to delete the methionine at position 52 or to replace it with a non-oxidizing amino acid and to re-insert the recombinant aprotinin variant into the DN15 viral vector backbone. The sequence for DN15 is disclosed in U.S. patent application Ser. No. 11/172,549. In addition, the viral expression vectors containing the leucine-52 aprotinin variant, pLSB 1820, and the glutamine-52 aprotinin variant, pLSB 1819, have been deposited with ATCC, as described in detail below.

N. bethamiana plants were infected with infectious transcript for each variant. 2 plants with each condition were harvested. Approximately 2 leaves above the inoculated leaf were harvested. 250 mM NaCl, 15 mM Ascorbic Acid, 0.1% SMB, and 5 mM EDTA was used for extraction buffer. Tissue mass averaged 10 grams (range from 10.03 to 10.42 g). 40 ml of buffer per 10 g tissue used or 1:4 ratio. Ground in smallest stainless steel blender for 30 seconds on high and filtered through 4 layers cheesecloth. Samples pH adjusted from around 4.25-4.5 to 4 with phosphoric acid then centrifuged at 9800 g for 20 minutes. Samples filtered through 2 layers of Miracloth. Volumes recovered around 40 ml. Plants were extracted and the aprotinin was purified using SP chromatography followed by reverse phase chromatography. Purified aprotinin was analyzed as follows:
Purity by SDS-PAGE
Identity by MALDI-TOF, MS
Potency by trypsin inhibition assay
Protein concentration was determined by absorbance
Specific activity was determined from the protein concentration and trypsin inhibition assay.

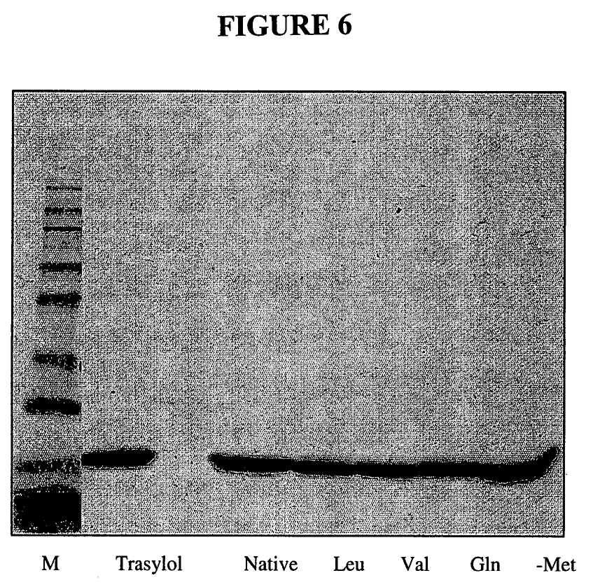

Samples were analyzed on Coomassie stained SDS-PAGE gels, as shown in FIG. 6. In addition, the activity of each construct was evaluated, as summarized in Table 7, below, and in FIG. 6.

TABLE 7

| | KIU/mg | Purity by | Relative rAprotinin | MALDI-TOF | MALDI-TOF |
|---|---|---|---|---|---|
| Aprotinin | Protein | SDS-PAGE | Expression | Theoretical | Measured |
| Trasylol | 7,116 | >99% | — | 6,512.5 | 6,511.8 |
| r-Aprotinin | 7,196 | >99% | 100 | 6,512.5 | 6,512.3 |
| r-Aprotinin-L | 7,147 | >99% | 100 | 6,493.5 | 6,494.3 |
| r-Aprotinin-Q | 7,218 | >99% | 100 | 6,509.5 | 6,510.7 |
| r-Aprotinin-V | 7,161 | >99% | 80 | 6,480.5 | 6,479.8 |
| r-Aprotinin-M | 6,847 | >99% | 25 | 6,381.3 | 6,381.9 |

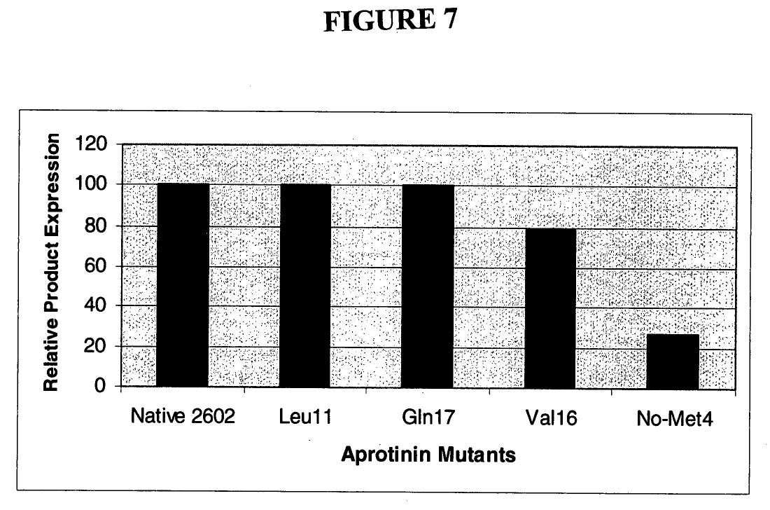

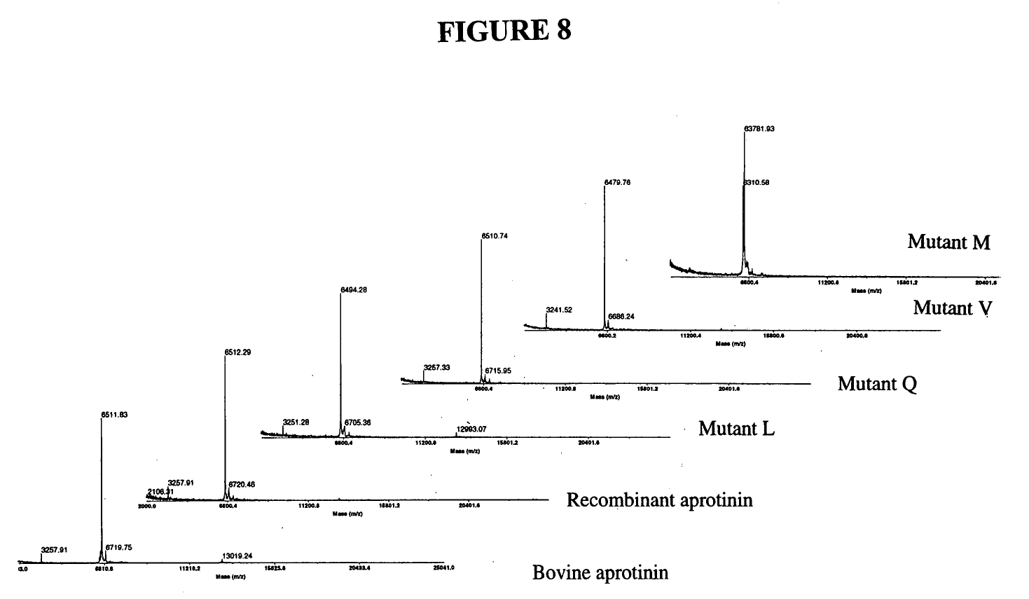

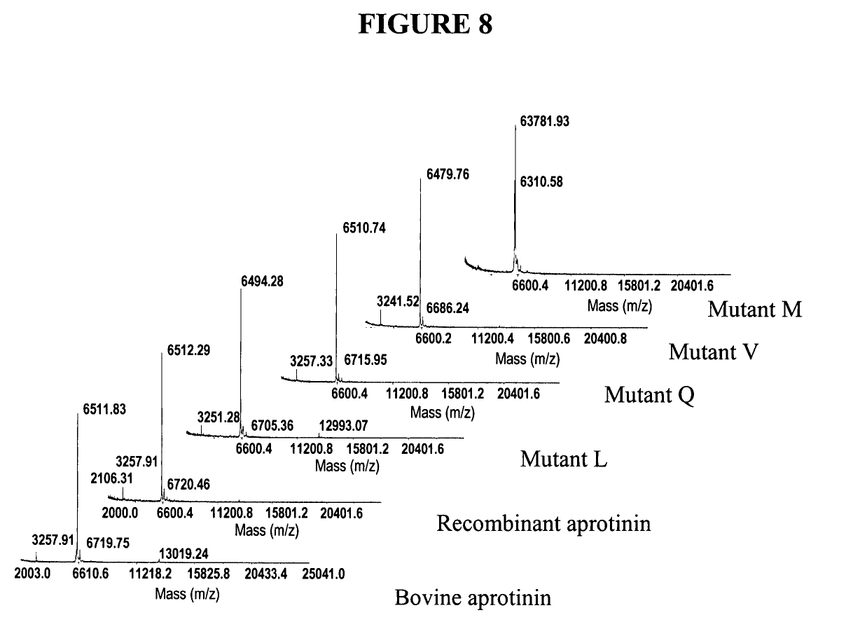

All of the methionine analogs produced the predicted size aprotinin protein, based upon MALDI-TOF, MS molecular mass data, as shown in Table 7, above and FIGS. 6 and 8. No evidence of oxidized aprotinin species were visible in the mass spectrums of the four aprotinin methionine analogs. In addition, the four r-Aprotinin analogs purified in a manner similar to the native r-Aprotinin (all greater than 99%) and had specific activities that were equivalent to r-Aprotinin and Trasylol, as shown in Table 7, above. The leucine and glutamine substitutions expressed aprotinin at a level equivalent to native r-Aprotinin whereas the valine substitution and minus methionine analogs expressed aprotinin levels that were 80% and 25% of the native sequence, respectively, as shown in FIG. 7 and Table 8, below.

These experiments indicate that methionine analogs can be produced using the GENEWARE® expression system and purified to a high level of purity. Mass spec data indicates that there is no oxidation occurring on the aprotinin methionine analogs. The r-Aprotinin analogs have equivalent trypsin inhibitory activity relative to native aprotinin. Level of aprotinin analog expression was comparable to the native enzyme and no major changes to an established extraction and purification protocol were required.

TABLE 8
Quantity One Analysis

| | ul Boiled | ul Sample | | | | mg in S1 | kg | |
|---|---|---|---|---|---|---|---|---|
| Sample | Sample | Loaded | Concentration | ug/ml | ml | sample | tissue | mg/kg |
| 2602 | 15 | 11.25 | 1342 | 119.2889 | 40 | 4.8 | 0.01 | 477 |
| Val | 9 | 6.75 | 311 | 46.07407 | 40 | 1.8 | 0.01 | 184 |
| Val | 15 | 11.25 | 745 | 66.22222 | 40 | 2.6 | 0.01 | 265 |
| Gln | 9 | 6.75 | 523 | 77.48148 | 40 | 3.1 | 0.01 | 310 |
| Gln | 15 | 11.25 | 1197 | 106.4 | 40 | 4.3 | 0.01 | 426 |
| Leu 10 | 15 | 11.25 | 1398 | 124.2667 | 40 | 5.0 | 0.01 | 497 |
| Leu 11 | 15 | 11.25 | 2210 | 196.4444 | 40 | 7.9 | 0.01 | 786 |
| Met | 15 | 11.25 | 247 | 21.95556 | 40 | 0.9 | 0.01 | 88 |

Deposit Information

The following plasmids were deposited under the terms of the Budapest Treaty with the American Type Culture Collection, 10801 University Blvd., Manassas, Va. 20110-2209, USA (ATCC):
Plasmid pLSB2602 is Patent Deposit PTA-6577, deposited Feb. 10, 2005.
Plasmid pLSB1819 is Patent Deposit PTA-6578, deposited Feb. 10, 2005.
Plasmid pLSB1820 is Patent Deposit PTA-6579, deposited Feb. 10, 2005.

These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit or 5 years after the last request, whichever is later. The assignee of the present application has agreed that if a culture of the materials on deposit should be found nonviable or be lost or destroyed, the materials will be promptly replaced on notification with another of the same. Availability of the deposited material is not to be construed as a license to practice the invention in contravention of the rights granted under the authority of any government in accordance with its patent laws, or as a license to use the deposited material for research.

Accordingly, the present invention has been described with some degree of particularity directed to the preferred embodiment of the present invention. It should be appreciated, though, that the present invention is defined by the following claims construed in light of the prior art so that modifications or changes may be made to the preferred embodiment of the present invention without departing from the inventive concepts contained herein.

### REFERENCES

1. Coperman, R. J., J. R. Hartman, and J. C. Watterson (1969) Phytopathology 59:1012.
2. Dawson, W. O., D. L. Beck, D. A., Knorr, and G. L. Grantham (1986) cDNA cloning of the complete genome of tobacco mosaic virus and production of infectious transcripts. Proc. Natl. Acad. Sci. (USA) 83:1832-1836.
3. Dawson, W. O. and Lehto, K. M. (1990) Regulation of tobamovirus gene expression. Ad. Virus Res. 38:307-342.
4. Dawson, W. O. (1992) Tobamovirus-Plant Interactions. Virology 186:359-367.
5. Deom, C. M., M. Lapidot, and R. N. Beachy (1992) Plant virus movement proteins. Cell 69:221-224.
6. Desnick, R. J., Y. A. Ioannou, and C. M. Eng (1995) α-Galactosidase A Deficiency: Fabry Disease, In: The Metabolic Bases of Inherited Diseases, C. R. Scriver, A. L. Beaudet, W. S. Sly, and D. Valle (eds.) McGraw-Hill, pp. 2741-2784.
7. de Zoeten, G. A. and G. Gaard. 1984. The presence of viral antigen in the apoplast of systemically virus-infected plants. Virus Res. 1:713-725.
8. Donson, J., C. M. Kearney, M. E. Hilf, and W. O. Dawson. (1991) Systemic expression of bacterial gene by a tobacco mosaic virus-based vector. Proc. Natl. Acad. Sci. (USA) 88:7204-7208.
9. Fitchen, J., R. N. Beachy, and M. B. Hein (1995) Plant virus expressing hybrid coat protein with added murine epitope elicits autoantibody response. Vaccine 13:1051-1057.
10. Fitzmaurice, W. P. (Large Scale Biology Corporation (2002). Interspecific Nicotiana Hybrids and Their Progeny. U.S. Pat. No. 6,344,597B1
11. Fritz, H., Hartwich, G., Werle, E., (1966) Hoppe-Seylers Zeitschrift Für Physiologishche Chemie (Berlin) 345, 150-167
12. Goldbach, R. and T. Hohn. (1997) Plant viruses as gene vectors. Meth. Plant. Biochem. 10b:103-120.
13. Gooding, G. V. and T. T. Herbert. (1967) A simple technique for purification of tobacco mosaic virus in large quantities. Phytopathology 57:1285.
14. Grill, L. K. (1992) 1991 Tobacco field trials report and soil and plant analysis follow-up on the 1991 tobacco field trials report; Filed with the USDA-APHIS, Hyattsville, Md.
15. Grill, L. K. (1993) Tobacco mosaic virus as a gene expression vector. Agro. Food Industy Hi Tech. November/December 20-23.
16. Hamamoto, H., Y. Sugiyama, N. Nadagawa, E. Hashida, Y. Matsunaga, S. Takemoto, Y. Watanabe, Y. Okada. (1993) A new tobacco mosaic virus vector and its use for the systemic production of angiotensin-1-converting enzyme inhibitor in transgenic tobacco and tomato. Bio/Techology 11:930-932.
17. Kassell, B. (1970) Methods in Enzymology XIX, 844-852
18. Kearney, C. M., J. Donson, G. E. Jones, and W. O. Dawson (1993) Low level of genetic drift in foreign sequences replicating in an RNA virus in plants. Virology 192:11-17.
19. Kermode, A. R. (1996) Mechanisms of intracellular protein transport and targeting in plant cells. Crit Rev. Plant Sc. 15:285-423.
20. Klement, Z. (1965) Method for obtaining fluid from the intercellular spaces of foliage and the fluid's merit as substrate for phytobacterial pathogens. Phytopathology 55:1033-1034.
21. Kumagai, M. H., T. H. Turpen, N. Winzettl, G. Della-Cioppa, A. M. Turpen, J. Donson, M. E. Hilf, G. L. Grantham, W. O. Dawson, T. P. Chow, M. Piatak, Jr., and L. K. Grill (1993) Rapid, high-level expression of biologically active α-trichosanthin in transfected plants by an RNA viral vector. Proc. Natl. Acad. Sci. (USA) 90:427-430.
22. Kumagai, M. H., J. Donson, G. Della-Cioppa, D. Harvey, K. Hanley, and L. K. Grill. (1995) Cytoplasmic inhibition of carotenoid biosynthesis with virus-derived RNA. Proc. Natl. Acad. Sci. (USA) 92:1679-1683.
23. Namba, K., R. Pattanayek, and G. Stubbs (1989) Visualization of protein-nucleic acid interactions in a virus: refined structure of intact tobacco mosaic virus at 2.9 A resolution by X-ray fiber diffraction. J. Mol. Biol. 208:307-325.
24. Pogue, G. P., T. H. Turpen, J. Hidalgo, T. I. Cameron, G. J., Murray, R.O. Brady, and L. K. Grill (1997) Production and purification of a highly active human ac-galactosidase A using a plant virus expression system. Abstract. Amer. Soc. Virol. Meeting. Bozeman, Mo.; p 162.
25. Pogue, G. P., Lindo, J. A., Garger, S. J., & Fitzmaurice, W. P. (2002). Making an Ally from an Enemy: Plant Virology and the New Agriculture. Annu. Rev. Phytopathol. 40, 45-74.
26. Scholthof, H. B., K. -B. G. Scholthof, and A. O. Jackson (1996) Plant virus gene vectors for transient expression of foreign proteins in plants. Annu. Rev. Phytopathol. 34:299-323.
27. Scopes, R. K. Protein Purification: Principles and Practice. 3rd ed. Springer Verlag, NY, N.Y.
28. Shivprasad, S., Pogue, G. P., Lewandowski, D. J., Hidalgo, J., Donson, J., Grill, L. K., Dawson, W. O. 1999. Heterologous sequences greatly affect foreign gene expression in tobacco mosaic virus-based vectors. Virology 255:312-323.
29. Siegal, A., V., Hari, and K. Kolacz (1978) The effect of tobacco mosaic virus infection on host and virus specific protein synthesis. Virology 85:494-503.
30. Sugiyama, Y., H. Hamamoto, S. Takemoto, Y. Watanabe, Y. Okada. (1995) Systemic production of foreign peptides on the particle surface of tobacco mosaic virus. FEBS Let. 359:247-250.
31. Turpen, T. H., S. J. Reinl, Y. Charoenvit, S. L. Hoffinan, V. Fallarme, and L. K. Grill. (1995) Malarial epitopes expressed on the surface of recombinant tobacco mosaic virus. Bio/Technology 13:53-57.
32. Turpen, T. H., T. I. Cameron, S. J. Reinl, G. P. Pogue, S. J. Garger, M. J. McCulloch, R. B. Holtz, and L. K. Grill (1997) Production of recombinant proteins in plants: Pharmaceutical applications. The Soc. Exper. Biol., Canterbury, U.K. J. Exp. Botany (Suppl.) 48:12.
33. Zaitlin, M. and H. W. Israel. (1975) Tobacco mosaic virus (type strain). C.M.I./A.A.B. Descriptions of Plant Viruses. Wm. Culross and Son, Ltd. UK.

What is claimed:

1. A composition comprising a plant produced recombinant variant aprotinin, wherein the variant aprotinin has an amino acid other than methionine at position 52.

2. The composition of claim 1 wherein the amino acid is selected from a group consisting of glutamine, leucine and valine.

3. The composition of claim 2 wherein the amino acid is glutamine.

4. The composition of claim 2 wherein the amino acid is leucine.

5. The composition of claim 2 wherein the amino acid is valine.

6. The composition of claim 1, wherein the composition is free of microbial and mammalian impurities.

7. An isolated DNA molecule, comprising
d. a DNA sequence encoding a variant aprotinin, wherein the variant aprotinin has an amino acid other than methionine at position 52; and
e. a DNA sequence encoding an RNA subgenomic promoter;
f. wherein the DNA sequence encoding aprotinin having a oxidation-resistant amino acid at position 52 is attached at its 5′ end to the DNA sequence encoding an RNA subgenomic promoter so that when a resulting negative sense RNA molecule encoded by the DNA molecule is present, then expression of the aprotinin-encoding DNA sequence is allowed in a plant.

8. An isolated RNA molecule, comprising:
d. a plus sense single stranded RNA subgenomic promoter sequence, and
e. a plus sense single stranded RNA sequence encoding a variant aprotinin, wherein the variant aprotinin has an amino acid other than methionine at position 52;
f. wherein the sequence encoding the variant aprotinin is linked at its 5′ end to said plus sense single stranded RNA subgenomic promoter sequence.

9. A recombinant single stranded plus sense plant viral RNA, comprising:
f. an RNA coding sequence for 126-kDa and 183-kDa replicase subunits;
g. a first coat protein subgenomic promoter sequence being attached at its 3′ end to an RNA sequence coding for a variant aprotinin, wherein the variant aprotinin has an amino acid other than methionine at position 52.

10. A recombinant single stranded plus sense plant viral RNA, further comprising:
h. the RNA coding sequence for 126-kDa and 183-kDa replicase subunits attached at its 3′ end to a 30-kDa open reading frame of a viral movement protein,
i. the 30-kDa open reading frame of a viral movement protein containing the first coat protein subgenomic promoter sequence;
j. the RNA sequence coding for variant aprotinin attached at its 3′ end to a second coat protein subgenomic promoter sequence;
k. the second coat protein subgenomic promoter sequence attached at its 3′ end to a coat protein coding sequence.

11. A recombinant cDNA plasmid, comprising a phage DNA dependent RNA polymerase promoter operably linked to a cDNA sequence encoding the recombinant single stranded plus sense plant viral RNA of claim 9.

12. A host plant cell transfected with at least one copy of the recombinant single stranded plus sense plant viral RNA of claim 9.

13. A host plant cell transfected with at least one copy of the recombinant single stranded plus sense plant viral RNA of claim 10.

14. A virus particle made in the host cell of claim 13.

15. A host plant transfected with the recombinant single stranded plus sense plant viral RNA of claim 10.

16. A host plant transfected with the virus particles of claim 14.

17. A cDNA plasmid according to claim 11 wherein the phage DNA dependent RNA polymerase promoter is a T7, SB6 or lambda phage promoter.

18. A plant-compatible expression vector comprising an artificial polynucleotide encoding a recombinant variant aprotinin, wherein the variant aprotinin has an amino acid other than methionine at position 52.

19. The plant-compatible expression vector of claim 18 wherein the vector is a plant viral vector.

20. A host plant cell comprising the expression vector of claim 19.

21. A process for producing a polypeptide comprising a variant aprotinin having an amino acid other than methionine at position 52 comprising transforming a plant with the expression vector of claim 18.

22. A process for producing a polypeptide comprising a variant aprotinin having an amino acid other than methionine at position 52 comprising infecting the plant with a viral vector comprising the expression vector of claim 18.


SEQUENCE LISTING (3 sequences)

SEQ ID NO: 1 - DNA, 255 bp, Artificial
  bovine aprotinin with extensin signal peptide
atgggaaaaatggcttctctatttgccacatttttagtggttttagtgtcacttagcttagctagcgaaagctccgccagaccggacttttgtttagagccaccttacactggtccatgtaaagcaagaataataagatacttctacaatgcgaaggctggattgtgtcaaactttcgtatacggaggttgtagagcaaaaaggaataatttcaagtctgcggaggactgtatgagaacttgtggaggtgcataa

SEQ ID NO: 2 - protein, 84 aa, Artificial
  Synthetic Construct
MGKMASLFATFLVVLVSLSLASESSARPDFCLEPPYTGPCKARIIRYFYNAKAGLCQTFVYGGCRAKRNNFKSAEDCMRTCGGA

SEQ ID NO: 3 - DNA, 9948 bp, Artificial
  Viral Vector Encoding Aprotinin, See Example 1 - pLSB2602
gtatttttacaacaattaccaacaacaacaaacaacagacaacattacaattactatttacaattacaatggcatacacacagacagctaccacatcagctttgctggacactgtccgaggaaacaactccttggtcaatgatctagcaaagcgtcgtctttacgacacagcggttgaagagtttaacgctcgtgaccgcaggcccaaggtgaacttttcaaaagtaataagcgaggagcagacgcttattgctacccgggcgtatccagaattccaaattacattttataacacgcaaaatgccgtgcattcgcttgcaggtggattgcgatctttagaactggaatatctgatgatgcaaattccctacggatcattgacttatgacataggcgggaattttgcatcgcatctgttcaagggacgagcatatgtacactgctgcatgcccaacctggacgttcgagacatcatgcggcacgaaggccagaaagacagtattgaactatacctttctaggctagagagaggggggaaaacagtccccaacttccaaaaggaagcatttgacagatacgcagaaattcctgaagacgctgtctgtcacaatactttccagacatgcgaacatcagccgatgcagcaatcaggcagagtgtatgccattgcgctacacagcatatatgacataccagccgatgagttcggggcggcactcttgaggaaaaatgtccatacgtgctatgccgctttccacttctccgagaacctgcttcttgaagattcatgcgtcaatttggacgaaatcaacgcgtgtttttcgcgcgatggagacaagttgaccttttcttttgcatcagagagtactcttaattactgtcatagttattctaatattcttaagtatgtgtgcaaaacttacttcccggcctctaatagagaggtttacatgaaggagtttttagtcaccagagttaatacctggttttgtaagttttctagaatagatacttttcttttgtacaaaggtgtggcccataaaagtgtagatagtgagcagttttatactgcaatggaagacgcatggcattacaaaaagactcttgcaatgtgcaacagcgagagaatcctccttgaggattcatcatcagtcaattactggtttcccaaaatgagggatatggtcatcgtaccattattcgacatttctttggagactagtaagaggacgcgcaaggaagtcttagtgtccaaggatttcgtgtttacagtgcttaaccacattcgaacataccaggcgaaagctcttacatacgcaaatgttttgtccttcgtcgaatcgattcgatcgagggtaatcattaacggtgtgacagcgaggtccgaatgggatgtggacaaatctttgttacaatccttgtccatgacgttttacctgcatactaagcttgccgttctaaaggatgacttactgattagcaagtttagtctcggttcgaaaacggtgtgccagcatgtgtgggatgagatttcgctggcgtttgggaacgcatttccctccgtgaaagagaggctcttgaacaggaaacttatcagagtggcaggcgacgcattagagatcagggtgcctgatctatatgtgaccttccacgacagattagtgactgagtacaaggcctctgtggacatgcctgcgcttgacattaggaagaagatggaagaaacggaagtgatgtacaatgcactttcagaattatcggtgttaagggagtctgacaaattcgatgttgatgttttttcccagatgtgccaatctttggaagttgacccaatgacggcagcgaaggttatagtcgcggtcatgagcaatgagagcggtctgactctcacatttgaacgacctactgaggcgaatgttgcgctagctttacaggatcaagagaaggcttcagaaggtgcattggtagttacctcaagagaagttgaagaaccgtccatgaagggttcgatggccagaggagagttacaattagctggtcttgctggagatcatccggaatcgtcctattctaagaacgaggagatagagtctttagagcagtttcatatggcgacggcagattcgttaattcgtaagcagatgagctcgattgtgtacacgggtccgattaaagttcagcaaatgaaaaactttatcgatagcctggtagcatcactatctgctgcggtgtcgaatctcgtcaagatcctcaaagatacagctgctattgaccttgaaacccgtcaaaagtttggagtcttggatgttgcatctaggaagtggttaatcaaaccaacggccaagagtcatgcatggggtgttgttgaaacccacgcgaggaagtatcatgtggcgcttttggaatatgatgagcagggtgtggtgacatgcgatgattggagaagagtagctgttagctctgagtctgttgtttattccgacatggcgaaactcagaactctgcgcagactgcttcgaaacggagaaccgcatgtcagtagcgcaaaggttgttcttgtggacggagttccgggctgtggaaaaaccaaagaaattctttccagggttaattttgatgaagatctaattttagtacctgggaagcaagccgcggaaatgatcagaagacgtgcgaattcctcagggattattgtggccacgaaggacaacgttaaaaccgttgattctttcatgatgaattttgggaaaagcacacgctgtcagttcaagaggttattcattgatgaagggttgatgttgcatactggttgtgttaattttcttgtggcgatgtcattgtgcgaaattgcatatgtttacggagacacacagcagattccatacatcaatagagtttcaggattcccgtaccccgcccattttgccaaattggaagttgacgaggtggagacacgcagaactactctccgttgtccagccgatgtcacacattatctgaacaggagatatgagggctttgtcatgagcacttcttcggttaaaaagtctgtttcgcaggagatggtcggcggagccgccgtgatcaatccgatctcaaaacccttgcatggcaagatcctgacttttacccaatcggataaagaagctctgctttcaagagggtattcagatgttcacactgtgcatgaagtgcaaggcgagacatactctgatgtttcactagttaggttaacccctacaccggtctccatcattgcaggagacagcccacatgttttggtcgcattgtcaaggcacacctgttcgctcaagtactacactgttgttatggatcctttagttagtatcattagagatctagagaaacttagctcgtacttgttagatatgtataaggtcgatgcaggaacacaatagcaattacagattgactcggtgttcaaaggttccaatctttttgttgcagcgccaaagactggtgatatttctgatatgcagttttactatgataagtgtctcccaggcaacagcaccatgatgaataattttgatgctgttaccatgaggttgactgacatttcattgaatgtcaaaaattgcatattggatatgtctaagtctgttgctgcgcctaaggatcaaatcaaaccactaatacctatggtacgaacggcggcagaaatgccacgccagactggactattggaaaatttagtggcgatgattaaaagaaactttaacgcacccgagttgtctggcatcattgatattgaaaatactgcatctttggttgtagataagttttttgatagttatttgcttaaagaaaaaagaaaaccaaataaaaatgtttctttgttcagtagagagtctctcaatagatggttagaaaagcaggaacaggtaacaataggccagctcgcagattttgattttgtggatttgccagcagttgatcagtacagacacatgattaaagcacaacccaaacaaaagttggacacttcaatccaaacggagtacccggctttgcagacgattgtgtaccattcaaaaaagatcaatgcaatattcggcccgttgtttagtgagcttactaggcaattactggacagtgttgattcgagcagatttttgtttttcacaagaaagacaccagcgcagattgaggatttcttcggagatctcgacagtcatgtgccgatggatgtcttggagctggatatatcaaaatacgacaaatctcagaatgaattccactgtgcagtagaatacgagatctggcgaagattgggtttcgaagacttcttgggagaagtttggaaacaagggcatagaaagaccaccctcaaggattataccgcaggtataaaaacttgcatctggtatcaaagaaagagcggggacgtcacgacgttcattggaaacactgtgatcattgctgcatgtttggcctcgatgcttccgatggagaaaataatcaaaggagccttttgcggtgacgatagtctgctgtactttccaaagggttgtgagtttccggatgtgcaacactccgcgaatcttatgtggaattttgaagcaaaactgtttaaaaaacagtatggatacttttgcggaagatatgtaatacatcacgacagaggatgcattgtgtattacgatcccctaaagttgatctcgaaacttggtgctaaacacatcaaggattgggaacacttggaggagttcagaaggtctctttgtgatgttgctgtttcgttgaacaattgtgcgtattacacacagttggacgacgctgtatgggaggttcataagaccgcccctccaggttcgtttgtttataaaagtctggtgaagtatttgtctgataaagttctttttagaagtttgtttatagatggctctagttgttaaaggaaaagtgaatatcaatgagtttatcgacctgacaaaaatggagaagatcttaccgtcgatgtttacccctgtaaagagtgttatgtgttccaaagttgataaaataatggttcatgagaatgagtcattgtcagaggtgaaccttcttaaaggagttaagcttattgatagtggatacgtctgtttagccggtttggtcgtcacgggcgagtggaacttgcctgacaattgcagaggaggtgtgagcgtgtgtctggtggacaaaaggatggaaagagccgacgaggccactctcggatcttactacacagcagctgcaaagaaaagatttcagttcaaggtcgttcccaattatgctataaccacccaggacgcgatgaaaaacgtctggcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccggaaaaaagagtgatgtccgcaaagggaaaaatagtagtagtgatcggtcagtgccgaacaagaactatagaaatgttaaggattttggaggaatgagttttaaaaagaataatttaatcgatgatgattcggaggctactgtcgccgaatcggattcgttttaaatagatcttacagtatcactactccatctcagttcgtgttcttgtcattaattaacatgggaaaaatggcttctctatttgccacatttttagtggttttagtgtcacttagcttagctagcgaaagctccgcccggcctgacttctgcctagagcctccatatacgggtccctgcaaggccagaattatcagatacttctacaacgccaaggctgggctctgccagacctttgtatatggcggctgcagagctaaaagaaacaatttcaagagcgcagaggactgcatgaggacctgtggtggtgcttagcctaggctcgaggggtagtcaagatgcataataaataacggattgtgtccgtaatcacacgtggtgcgtacgataacgcatagtgtttttccctccacttaaatcgaagggttgtgtcttggatcgcgcgggtcaaatgtatatggttcatatacatccgcaggcacgtaataaagcgaggggttcgggtcgaggtcggctgtgaaactcgaaaaggttccggaaaacaaaaaagagagtggtaggtaatagtgttaataataagaaaataaataatagtggtaagaaaggtttgaaagttgaggaaattgaggataatgtaagtgatgacgagtctatcgcgtcatcgagtacgttttaatcaatatgccttatacaatcaactctccgagccaatttgtttacttaagttccgcttatgcagatcctgtgcagctgatcaatctgtgtacaaatgcattgggtaaccagtttcaaacgcaacaagctaggacaacagtccaacagcaatttgcggatgcctggaaacctgtgcctagtatgacagtgagatttcctgcatcggatttctatgtgtatagatataattcgacgcttgatccgttgatcacggcgttattaaatagcttcgatactagaaatagaataatagaggttgataatcaacccgcaccgaatactactgaaatcgttaacgcgactcagagggtagacgatgcgactgtagctataagggcttcaatcaataatttggctaatgaactggttcgtggaactggcatgttcaatcaagcaagctttgagactgctagtggacttgtctggaccacaactccggctacttagctattgttgtgagatttcctaaaataaagtcactgaagacttaaaattcagggtggctgataccaaaatcagcagtggttgttcgtccacttaaatataacgattgtcatatctggatccaacagttaaaccatgtgatggtgtatactgtggtatggcgtaaaacaacggaaaagtcgctgaagacttaaaattcagggtggctgataccaaaatcagcagtggttgttcgtccacttaaaaataacgattgtcatatctggatccaacagttaaaccatgtgatggtgtatactgtggtatggcgtaaaacaacggagaggttcgaatcctcccctaaccgcgggtagcggcccaggtacccggatgtgttttccgggctgatgagtccgtgaggacgaaacctggctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttgcgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcaagcttaatacgactcactata